(19) United States
(12) Patent Application Publication
(10) Pub. No.: US 2003/0017985 A1
(43) Pub. Date: Jan. 23, 2003

(54) Molecules that influence pathogen resistance

(76) Inventors: Gregory Alan Taylor, Durham, NC (US); George F. Vande Woude, Ada, MI (US)

Correspondence Address: KLARQUIST SPARKMAN, LLP, 121 SW SALMON STREET, SUITE 1600, PORTLAND, OR, 97204, US

(21) Appl. No.: 10/169,315

(22) PCT Filed: Jan. 2, 2001
(86) PCT No.: PCT/US01/00059

(51) Int. Cl. A61K 38/17

(52) U.S. Cl. 514/12

(57) ABSTRACT

The functions of IFNγ-induced GTPases of the IGTP-family as strong anti-infective agents, and more particularly a strong anti-parasite and/or anti-bacterial agents, are disclosed. These molecules (in both protein and nucleic acid forms) are effective to modify anti-microbial e.g., anti-bacterial and/or anti-parasitic) immune responses in a subject, to prevent or inhibit replication or infectivity of microbe, to treat microbial diseases, and to detect susceptibility of a subject to microbial infection. This invention also provides kits and compounds useful in such methods. Also provided are transgenic non-human animals in which IGTP-family member gene expression has been altered, and the use of such animals to screen for anti-microbial agents.

### FIELD

The present invention relates to the field of prevention and treatment of infectious diseases through modification of immune response(s). In particular, it relates to the involvement of GTPase molecule(s), particularly IFN-γ-inducible GTPases, in immune responses to infectious disease, such as bacterial and parasitic (e.g., protozoan) disease.

### BACKGROUND

Interferon γ (IFNγ) is an important cytokine for control of infectious agents and regulation of the immune system (Stark et al., Annu. Rev. Biochem., 67:227-264, 1998; Boehm et al., Annu. Rev. Immunol., 15:749-795, 1997; Billiau, Adv. Immunol., 62:61-130, 1996). Mice that lack IFNγ or the IFNγ receptor have decreased immune response to parasites, bacteria, viruses, and tumors (Kaplan et al., Pro. Natl. Acad. Sci., 95:7556-7561, 1998; Dalton et al., Science, 259:1739-1742, 1993; Huang et al., Science, 259:1742-1745, 1993).

IFNγ regulates expression of over 200 genes that are thought to mediate its effects (Der et al., Proc. Natl. Acad. Sci. USA, 95:15623-15628, 1998); however for many of these genes, their contribution to host defense is unknown. Recently, a new family of IFNγ-induced genes has been identified that includes at least six members: IGTP (Taylor et al., J. Biol. Chem., 271:20399-20405, 1996), LRG-47 (Sorace et al., J. Leukoc. Biol., 58:477484, 1995), IRG47 (Gilly & Wall, J. Immunol., 148:3275-3281, 1992), TGTP/Mg21 (Carlow et al., J. Immunol., 154:1724-1734, 1995; LaFuse et al., J. Leukoc. Biol., 57:477-483, 1995), IIGP (Boehm et al., J.Immunol., 161:6715-6723, 1998), and GTPI (Boehm et al., J.Immunol., 161:6715-6723, 1998). The expression of each of these genes is markedly increased by IFNγ in hematopoietic and non-hematopoietic cells, and each encodes a 47-48 kDa protein that contains GTP-binding sequences (Taylor et al., J. Biol. Chem., 271:20399-20405, 1996; Sorace et al., J. Leukoc. Biol., 58:477-484, 1995; Gilly & Wall, J. Immunol., 148:3275-3281, 1992; Carlow et al., J. Immunol., 154:1724-1734, 1995; LaFuse et al., J. Leukoc. Biol., 57:477-483, 1995; Boehm et al., J.Immunol., 161:6715-6723, 1998). IGTP is expressed at high levels in many IFNγ-stimulated cells including immune cells such as macrophages, T cells, and B cells, and nonimmune cells such as fibroblasts and hepatocytes (Taylor et al., J. Biol. Chem., 271:20399-20405, 1996).

Several of these proteins, including IGTP, LRG-47, and TGTP, have been shown to localize to the endoplasmic reticulum of cells; therefore, it has been suggested that the protein family may regulate expression and trafficking of proteins of immunological importance (Taylor et al., J. Biol. Chem., 272:10639-10645, 1997). While the family members display a high degree of homology overall they may be divided into two subgroups based on primary amino acid sequences, with the first subgroup including IGTP, LRG-47, and GTPI, and the second including IRG-47, TGTP/Mg21, and IIGP (Boehm et al., J.Immunol., 161:6715-6723, 1998).

The functions of the proteins in this GTPase family are unknown.

The immune mechanism(s) that regulate resistance to infectious agents, including bacterial, viral and protozoan infections (such as those of Toxoplasma gondii), are poorly understood. T. gondii is an intracellular protozoan parasite found throughout the world and capable of infecting all species of mammals and all types of cells within a given individual. The parasite can be contracted through ingestion of oocysts (mature T. gondii sexual-cycle cells) that are shed in the feces of infected cats, or through ingestion of undercooked meat that contains T. gondii cysts.

The parasite is extremely widespread. In the U.S.A., serological studies indicate that as many as 10-50% of the population has had contact with the parasite. In countries where eating lightly cooked or raw meat is more common, this figure can rise to as much as 85% (e.g., in France). Infection by the T. gondii organism leads to toxoplasmosis. In healthy adults, the disease is typically mild, producing few if any symptoms. In immunocompromised adults (e.g., those suffering from neoplastic disease or acquired immnunodeficiency syndrome (AIDS), or undergoing post-transplantation therapy), however, the parasite can cause severe pathology.

In humans, toxoplasmosis pathology has traditionally been associated with the developing fetus, in which toxoplasmosis can cause severe neurological problems manifesting as hydrocephaly, mental retardation and/or blindness. Infection of the fetus during pregnancy will (in approximately 10% of the cases) lead to neonatal death or a severely multi-handicapped child, but in 90% of the cases the child will be bom with an asymptotic, latent infection. Up to 85% of patients with latent congenital toxoplasmosis will develop significant sequelae, including one or more episodes of active retinochoroiditis. Other clinical symptoms include inflammation, lymphadenitis, encephalitis and fever.

It is thus apparent that toxoplasmosis is a serious clinical concern, particularly in the case of AIDS patients and pregnant women. Current treatments are limited by adverse drug effects (Porter and Sande, New Engl. J. Med., 327:1643-1648, 1992). There therefore exists a continuing need for safe and effective anti-parasitic agents, and in particular anti-protozoan (e.g., Toxoplasma) agents.

Infectious diseases, and bacterial and parasitic diseases in general, are serious worldwide public health concerns. Parasitic infections are the most common infections in the world, both in humans and livestock, and are a significant cause of mortality and morbidity. There remains an unfulfilled need to provide effective preventative and therapeutic treatments to combat infections diseases, including protozoan and helminth parasitic and bacterial infectious disease.

### SUMMARY OF THE DISCLOSURE

It has now been found that IGTP-family proteins (IFN-γ inducible GTPases) mediate the immune response of mammals to specific infectious pathogens, and particularly that individual GTPases of this family are involved to greater or lesser extents in specific responses. More particularly, IGTP appears to be especially important in the development of immnunogenic resistance to protozoa, such as T. gondii in the acute response phase, while IRG-47 appears to be to be more important in resistance in the chronic stage of this infection. In contrast, LRG-47 appears to be important in host resistance to both bacterial and protozoan infections. Further, both IGTP and LRG-47 are involved in host response to lipopolysaccharide (LPS) induced shock, and are believed to dampen the immune response to LPS, thereby decreasing the likelihood of toxic shock during acute gram-negative bacterial infection.

This disclosure presents methods of modifying the immune response in a subject, particularly anti-bacterial and/or anti-parasite immunity, by modifying the activity of an IGTP-family protein or a related protein in the subject. Such modification can be through an increase or decrease in the level of IGTP-family protein expression, for instance by expression of a recombinant IGTP-family protein-encoding nucleic acid, or by administration to the subject of an IGTP-family protein, or a fragment, variant, analog, derivative or mimetic thereof that maintains immune response modifying activity. These methods can be used to treat subjects that are infected with or at risk for infection with an infectious biological agent, for instance a virus, a bacterium or a parasite.

The foregoing and other objects, features, and advantages of the disclosure will become more apparent from the following detailed description of particular embodiments which proceeds with reference to the accompanying drawings.

### BRIEF DESCRIPTION OF THE DRAWINGS

FIG. 1 is a schematic drawing of the replacement vector used to knock-out the IGTP gene, and a western analysis of protein produced from an IGTP knock-out mouse.

An IGTP replacement targeting vector was prepared from the indicated IGTP gene fragments and used to generate IGTP-deficient mice (FIG. 1A). Thymic protein from mice of the indicated genotypes was resolved by 10% SDS-PAGE, and used for western blotting with anti-C-terminal IGTP antisera (FIG. 1B). The positions of selected molecular weight markers are shown at the left.

FIG. 2 is a digital image of a western blot, showing the induction of IGTP expression by various pathogens, including bacteria (FIG. 2A), viruses (FIG. 2B), and parasitic protozoa (FIG. 2C).

Pairs of wild-type mice (FIG. 2B & FIG. 2C), or wild-type and IGTP-deficient mice (FIG. 2A), were inoculated with (FIG. 2A) 1000 Listeria monocytogenes for 3 days, (FIG. 2B) 10⁵ pfu MCMV for 36 or 72 hours, or (FIG. 2C) twenty cysts ME49 strain Toxoplasma gondii for 5 days. Protein lysates were prepared from the indicated tissues or cells and used for western blotting with an anti-IGTP antibody. The positions of selected molecular weight markers are indicated at the left.

FIG. 3 shows several graphs that indicate the differential susceptibility of IGTP-deficient mice to pathogens, and illustrates that IGTP-deficient mice are unusually susceptible to T. gondii infection.

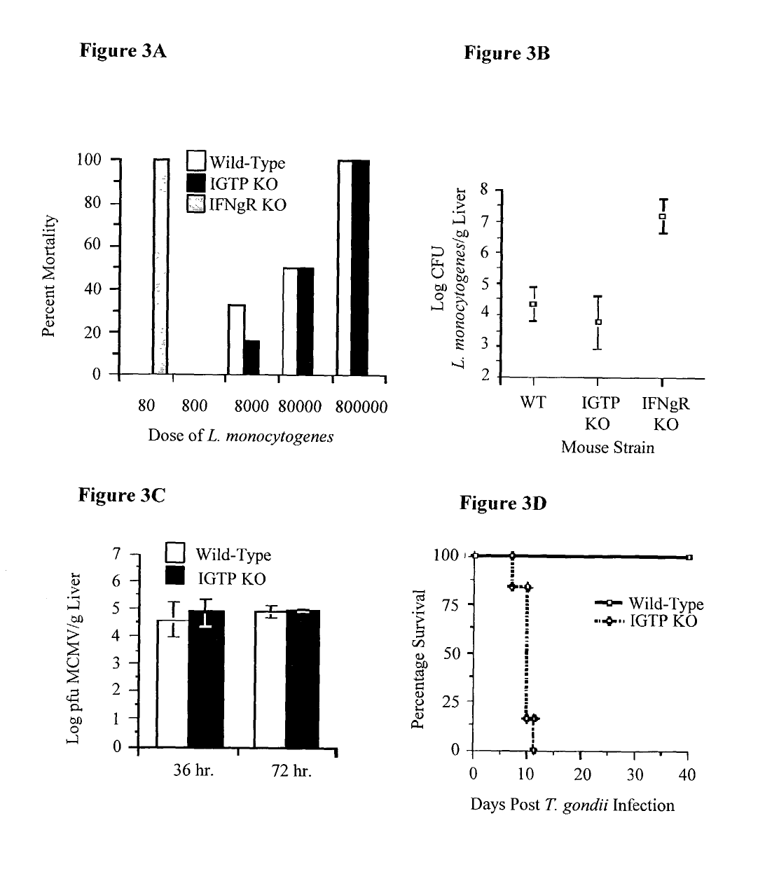

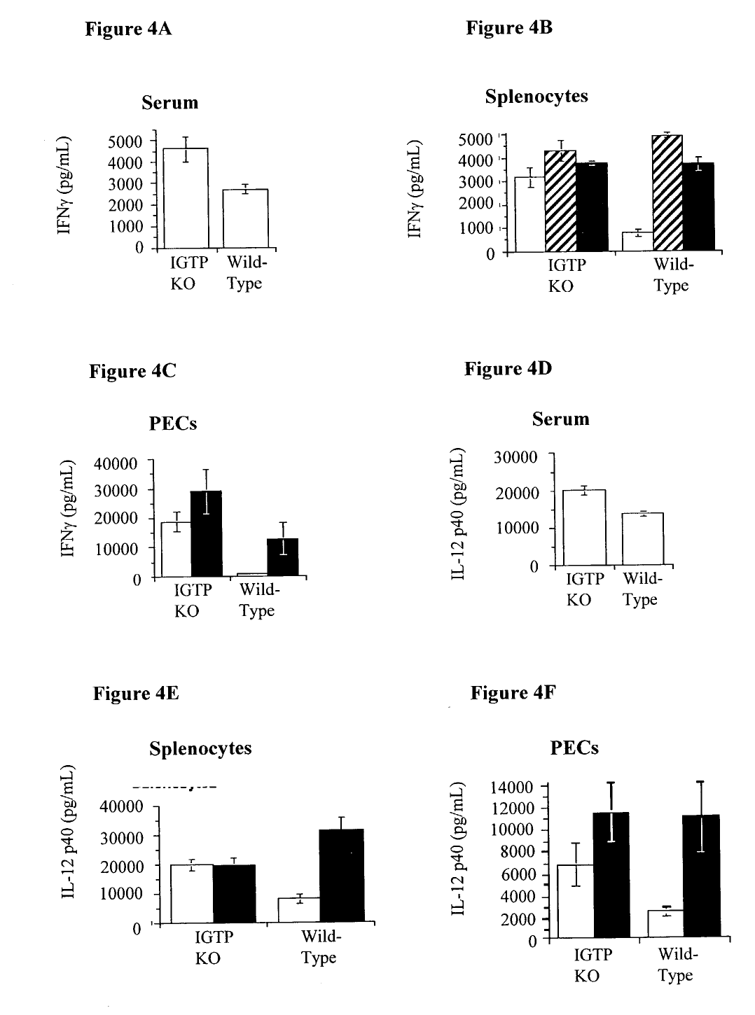

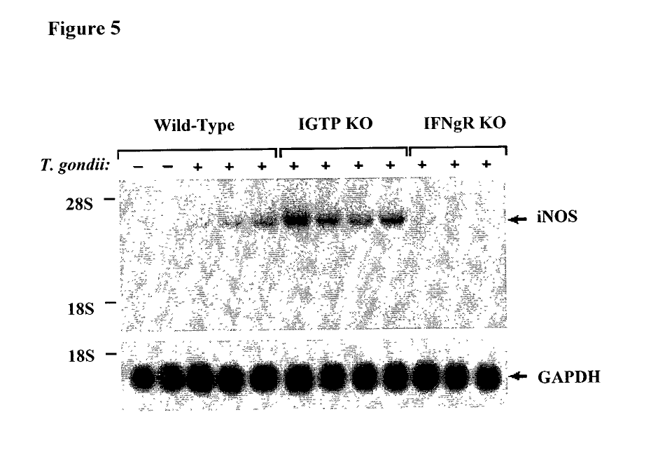

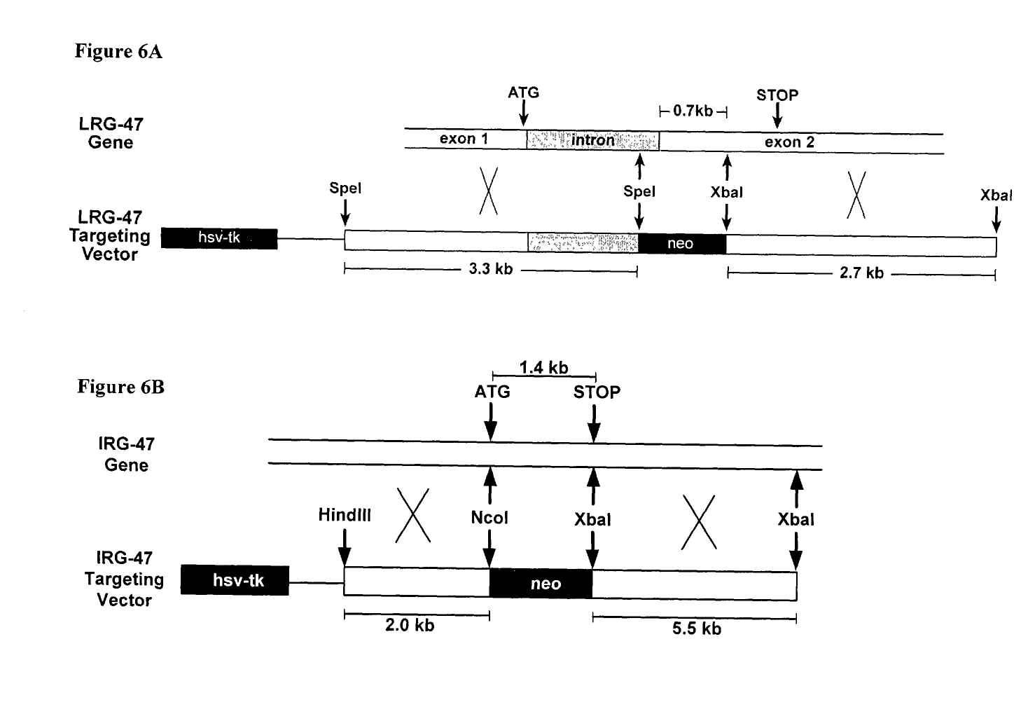

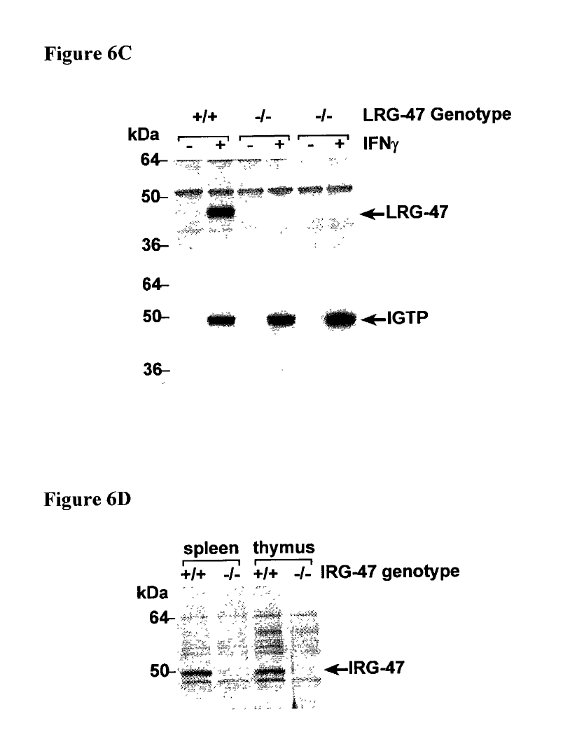

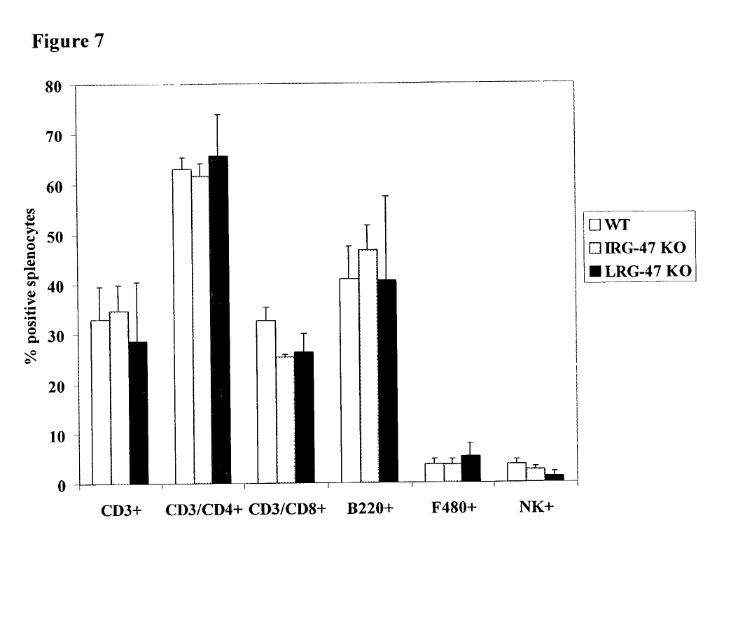

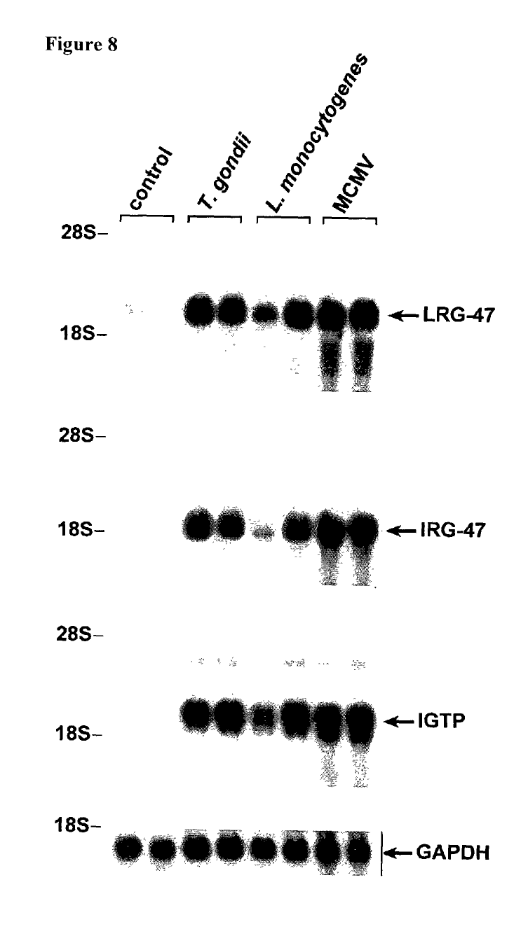

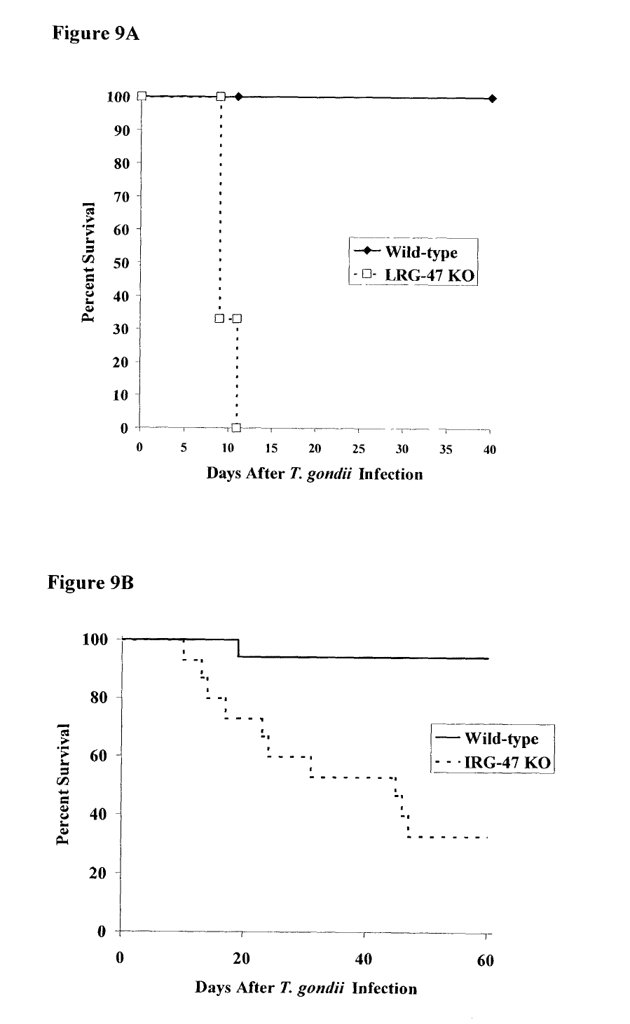

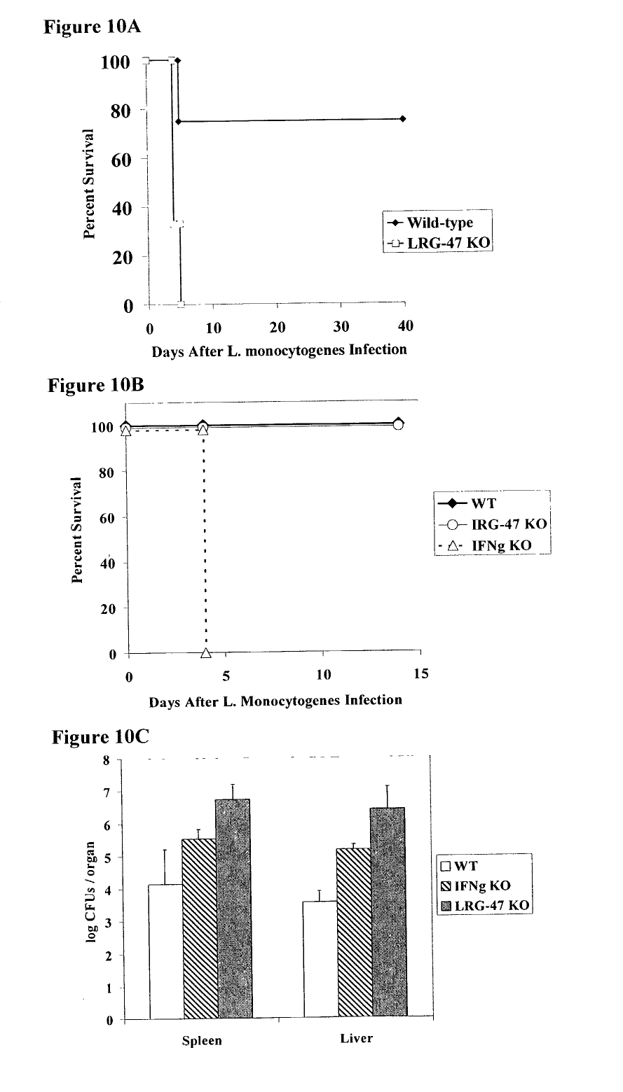

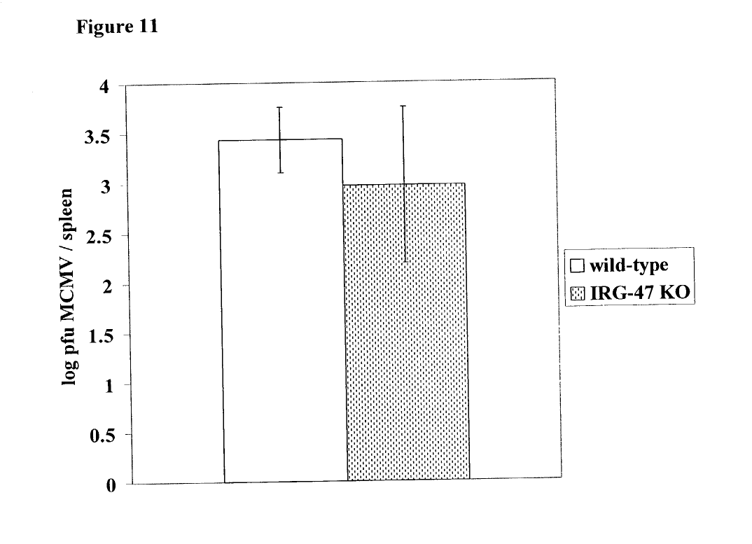

Wild-type, IGTP-deficient, or IFNγ receptor-deficient mice were inoculated i.p. with the indicated doses of L. monocytogenes, and mortality was assessed over a 14 day period (FIG. 3A). Wild-type, IGTP-deficient, or IFNγ receptor-deficient mice were inoculated i.p. with 1000 L. monocytogenes; after three days, the number of bacteria in the liver was determined (FIG. 3B). Wild-type or IGTP-deficient mice were inoculated with 10⁵ pfu MCMV i.p.; after 36 or 72 hours, the number of virus in the liver was determined (FIG. 3C). Wild-type and IGTP-deficient mice were inoculated i.p. with 20 cysts ME49 strain T. gondii. The cumulative mortality of the indicated numbers of IGTP-deficient mice (diamonds) or wild-type mice (squares) was followed for 40 days after infection (FIG. 3D). The graph is representative of three experiments.

FIG. 4 shows that production of IL-12 p40 and IFNγ is not diminished in T. gondii-infected IGTP-deficient mice.

Mice were inoculated i.p. with twenty cysts ME49 strain T. gondii. Five days post-inoculation of IGTP-deficient or wild-type mice, IL-12 p40 and IFNγ and levels were determined in sera (FIG. 4A, FIG. 4D), and the conditioned media of cultures splenocytes (FIG. 4B, FIG. 4E) or peritoneal exudate cells (PECs) (FIG. 4C, FIG. 4F). Splenocytes and PECs were incubated in control medium (open bars), medium supplemented with anti-CD3 antibody (striped bars), or medium supplemented with a soluble Toxoplasma antigen mixture, STAg (solid bars). Standard deviations for groups of four mice are shown.

FIG. 5 shows that production of inducible nitric oxide synthase (iNOS) is undiminished in T. gondii-infected IGTP-deficient mice.

Wild-type, IGTP-deficient, and IFNγR-deficient mice were inoculated i.p. with 20 cysts ME49 strain T. gondii. Eight days post-inoculation, hepatic mRNA was prepared and used for sequential northern blotting with iNOS and GAPDH probes. Positions of the major nbosomal RNA species are indicated as determined from the stained gel.

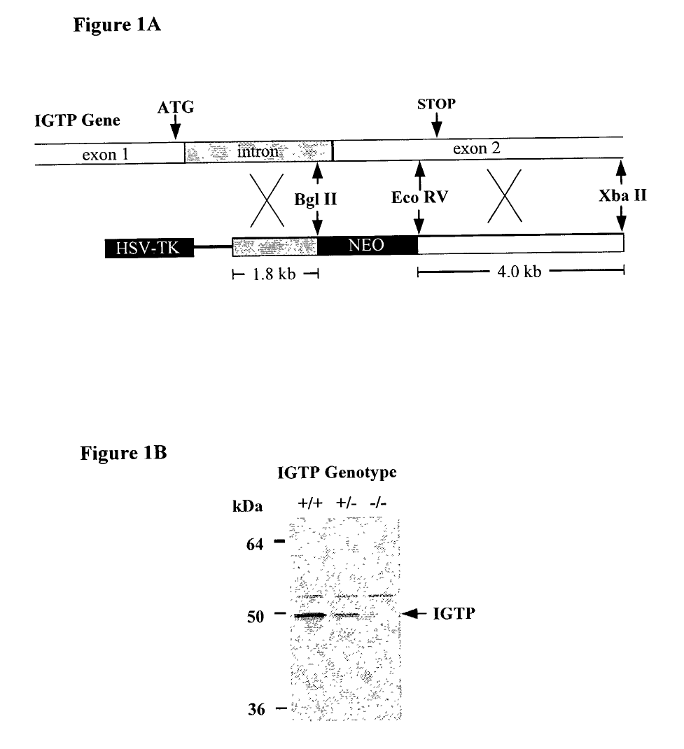

FIG. 6 shows the construction of LRG-47 (FIG. 1A) and IRG-47 (FIG. 1B) knock out constructs, and Western blot characterization of the resulting protein-deficient mice (FIGS. 1C and 1D, respectively).

FIG. 7 is a bar graph of the quantitative results of FACS analysis of splenocytes from adult LRG-47 and IRG-47-deficient mice. The graph shows that LRG-47 and IRG-47 deficient mice display no significant changes in the development of T cell, B cell, macrophage, and natural killer cell populations.

FIG. 8 is a series of Western blots using antibodies against LRG-47, IRG-47, and IGTP. Expression of each of these IGTP-family proteins is markedly increased following infection with each of the indicated pathogens. GADPH is provided as a control.

FIG. 9 is a pair of graphs, comparing the survival rate of wild-type versus LRG-47-deficient (FIG. 9A) and IRG-47-deficient (FIG. 9B) mice after infection with T. gondii. LRG-47-deficient mice are susceptible to acute T. gondii infection (dying 9-11 days post infection) (FIG. 9A), while IRG-47-deficient mice show only marginally reduced resistance with death mainly occurring during the chronic phase, between 1047 days post infection (FIG. 9B).

FIG. 10 is a series of three graphs, comparing the response of wild-type, IRG-47-deficient, LRG-47-deficient, and IFNγ-deficient mice to challenge with L. monocytogenes. FIG. 10A shows that LRG-47-deficient mice are highly susceptible to L. monocytogenes infection. This response is similar to that seen in IFNγ-deficient mice (FIG. 10B). In marked contrast, IRG-47-deficient mice showed no adverse effects (FIG. 10B), and survived for the full 40-day observation period. FIG. 10C is a bar graph showing that bacterial load was higher in both the liver and spleen of LRG-47-deficient mice than in wild-type mice, while IFNγ-deficient mice showed intermediate levels of bacterial load.

FIG. 11 is a bar graph, showing that comparable numbers of viral plaque-forming units (pfu's) were detected in wild-type and IRG-47-deficient mice after infection with MCMV.

### DETAILED DESCRIPTION

I. Abbreviations and Explanation of Terms

| A. | Abbreviations |
|---|---|
| cDNA: | complementary DNA |
| DNA: | deoxyribonucleic acid |
| ER: | endoplasmic reticulum |
| FACS: | fluorescent automatic cell sorter |
| IFN: | interferon (such as interferon-gamma, or IFNγ) |
| IGTP: | inducibly expressed GTPase protein |
| IL: | interleukin (such as interleukin-12, or IL-12) |
| iNOS: | inducible nitric oxide synthase |
| MCMV: | murine cytomegalovirus |
| NO: | nitric oxide |
| ORF: | open reading frame |
| PCR: | polymerase chain reaction |
| PECs: | peritoneal exudate cells |

B. Explanation of Terms

Unless otherwise noted, technical terms are used according to conventional usage. Definitions of common terms in molecular biology may be found in Benjamin Lewin, Genes V, published by Oxford University Press, 2000 (ISBN 0-19-879276-X); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanation of terms is provided:

Animal: Living multi-cellular vertebrate organisms, a category that includes, for example, mammals and birds. The term mammal includes both human and non-human mammals. Similarly, the term “subject” includes both human and veterinary subjects.

Antisense, Sense, and Antigene: Double-stranded DNA (dsDNA) has two strands, a 5′→3′ strand, referred to as the plus strand, and a 3′→5′ strand (the reverse compliment), referred to as the minus strand. Because RNA polymerase adds nucleic acids in a 5′→3′ direction, the minus strand of the DNA serves as the template for the RNA during transcription. Thus, the RNA formed will have a sequence complementary to the minus strand and identical to the plus strand (except that U is substituted for T).

Antisense molecules are molecules that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a dsDNA target

Binding or stable binding: An oligonucleotide binds or stably binds to a target nucleic acid if a sufficient amount of the oligonucleotide forms base pairs or is hybridized to its target nucleic acid, to permit detection of that binding. Binding can be detected by either physical or functional properties of the target:oligonucleotide complex. Binding between a target and an oligonucleotide can be detected by any procedure known to one skilled in the art, including both functional and physical binding assays. Binding may be detected functionally by determining whether binding has an observable effect upon a biosynthetic process such as expression of a gene, DNA replication, transcription, translation and the like.

Physical methods of detecting the binding of complementary strands of DNA or RNA are well known in the art, and include such methods as DNase I or chemical footprinting, gel shift and affinity cleavage assays, Northern blotting, dot blotting and light absorption detection procedures. For example, one method that is widely used, because it is so simple and reliable, involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate or melt.

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

cDNA: A piece of DNA lacking internal, non-coding segments (introns) and regulatory sequences which determine transcription. cDNA is synthesized in the laboratory by reverse transcription from messenger RNA extracted from cells.

Complex (complexed): Two proteins, or fragments or derivatives thereof, are said to form a complex when they measurably associate with each other in a specific manner. Such association can be measured in any of various ways, both direct and indirect. Direct methods may include co-migration in non-denaturing fractionation conditions, for instance. Indirect measurements of association will depend on secondary effects caused by the association of the two proteins or protein domains. For instance, the formation of a complex between a protein and an antibody may be demonstrated by the antibody-specific inhibition of some function of the target protein.

Deletion: The removal of a sequence of DNA, the regions on either side being joined together.

DNA: A long chain polymer that comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) in DNA molecules encode individual amino acid residues in a polypeptide. The term codon is also used for the corresponding (and complementary) sequences of three nucleotides in the mRNA into which the DNA sequence is transcribed.

Genetic fragment: Any polynucleotide, DNA or RNA, derived from a larger polynucleotide.

Homologs: Two nucleotide or amino acid sequences that share a common ancestral sequence and diverged when a species carrying that ancestral sequence split into two species. Homologs frequently show a substantial degree of sequence identity.

Hybridization: DNA molecules and nucleotide sequences which are derived from the disclosed DNA molecules as described above may also be defined as DNA sequences which hybridize under stringent conditions to the DNA sequences disclosed, or fragments thereof.

IGTP-family protein(s): A recently identified family of about 47 to about 48 kDa molecular weight, IFNγ-induced GTPases that are expressed in various cell types in mammals. Representative members of the IGTP-family of GTPases include IGTP (Accession U53219), GTP2/Mg21/TGTP (Accession U15636), IRG-47 (Accession M63630), LRG-47 (Accession U19119), GTP1 (Accession AJ007972), and IIGP (Accession AJ007971). As used herein, the term “IGTP-family protein” also encompasses fragments (portions of the full-length protein), variants (proteins in which the amino acid sequence has been altered through genetic or other techniques, or through natural mutation), analogs (non-protein organic molecules), derivatives (chemically functionalized protein molecules obtained starting with the disclosed protein sequences) or mimetics (three-dimensionally similar chemicals) of the native IGTP protein structure, as well as proteins sequence variants or genetic alleles, that maintain the ability to functionally modify an immune response, for instance an anti-microbial immune response, e.g. anti-parasitic or anti-bacterial.

Such molecules can be screened for such ability (such as generally an “IGTP-family activity” or an “[IGTP]-like activity,” where the reference is to an activity specific for one member of the IGTP-family) by assaying a protein similar to the disclosed IGTP-family protein, in that it has one or more conservative amino acid substitutions, or analogs, derivatives or mimetics thereof, and determining whether the similar protein, analog, derivative or mimetic provides immune modulatory effect, for instance anti-parasitic activity. It is possible, for instance, to determine such an activity by expressing the IGTP-family protein, fragment, variant, derivative, analog or mimetic in an animal otherwise deficient for that IGTP-family protein (e g., wherein the native IGTP gene has been knocked-out), and determining the change in response of the animal to an infectious challenge, such as a protozoan parasite, viral, or bacterial infection. In an alternative method, the IGTP-family protein-related compound is administered to the animal through some other method including those disclosed herein, and the change in immune response is once again determined.

In certain embodiments, such activity will include GTPase activity, and as such the GTPase activity of the protein, analog, derivative or mimetic can be measured directly, for instance using standard enzyme activity assay (see, e.g., Taylor et al., J. Biol. Chem., 271:20399-20405, 1996) or assay for guanine nucleotide binding (Taylor et al., J. Biol. Chem., 272, 10638-10645, 1997). The GTPase activity of these derivative compounds can be measured by any known means, including those discussed in this application.

IGTP-family protein encoding sequence or nucleic acid: A nucleic acid that encodes a protein member of the IGTP family of proteins.

Infectious agent: Bacterial, viral, fungal and parasitic (including helminthic and protozoan parasites) organisms that have the ability to infect subjects, for instance an animal and more particularly a mammal. A general discussion of infectious diseases and the organisms that cause them can be found in myriad references that are well known to those of skill in the art, for instance, Cecil Textbook of Medicine, Wyngaarden et al. (ed.), W. B. Saunders Co., Philadelphia, Pa., 1992, particularly Parts XX, XXI, and XXII.

Bacterial infectious organisms include, but are in no way limited to, Streptococcus sp., Haemophilus influenzae, Klebsiella sp., Escherichia sp., Legionella sp., Mycoplasma sp., Pneumocystis carinii, Listeria, Corynebacterium sp., Staphylococcus sp., Serratia, Pseudomonas, Shigella, Vibrio, Hemophilus sp., Yersinia, and Enterobacter, and include also diseases due to Mycobacteria, such as tuberculosis (caused by Mycobacterlum tuberculosis) and leprosy (caused by Mycobacterium leprae). Infectious Chlamydiae organisms are also encompassed in this definition (such as C. trachomatis), as are rickettsiae organisms (such as those causing typhus and potted fever). See, Cecil Textbook of Medicine, Wyngaarden et al. (ed.), W. B. Saunders Co., Philadelphia, Pa., 1992, pages 1608-1798.

Viral infectious organisms include viruses of the following families: Poxviridae, Herpesviridae, Adenoviridiae, Papovaviridae, Hepadnaviridae, Parvoviridae, Reviridae, Togoviridae, Flaviviridae, Coronaviridae, Paramyxoviridae, Rhabdoviridae, Bunyaviridae, Arenaviridae, Retroviridae, Picornaviridae, and Caliciviridae. Viral diseases and viruses include, but are in no way limited to, smallpox, Vaccinia, herpes sp., Influenza sp., Varicella-zoster, cytomegalovirus sp., Epstein-Barr disease, rubella, yellow fever, rabies, measles, Ebola, polio, and HIV (the cause of AIDS). See, Cecil Textbook of Medicine, Wyngaarden et al.. (ed.), W. B. Saunders Co., Philadelphia, Pa., 1992, pages 1798-1886.

Fungal infectious organisms (mycoses) include, but are in no way limited to Aspergillus sp., Candida sp., Cryptococcus neofonnans, Coccidiodes inmitis, Blastomyces dermatitidis, Rhizopus sp., Mucor sp., and Fusariaum sp. See, Cecil Textbook of Medicine, Wyngaarden et al. (ed.), W. B. Saunders Co., Philadelphia, Pa., 1992, pages 1886-1907.

Parasitic organisms include, but are in no way limited to, those organisms responsible for protozoan infections, e.g., toxoplasmosis (Toxoplasma sp.), malaria (Plasmodium sp.), sleeping sickness and Chaga's disease (trypanosomiasis; Tryapanosoma sp.), leishmaniasis (Leishmania sp.), cryptosporidiosis (Cryptosporidium sp.), giardiasis (Giardia sp.), amebiasis (Entamoeba sp.), or trichomoniasis (Trichomonas sp.), cestode infections (tapeworms (Diphyllobothrium latum), or echinococcosis (Echinococcosis sp.)), worm (Schistosoma sp.), fluke or nematode infections, or filariasis (various filarial parasites including Wuchereria bancrofti, Brugia malayi and Onchocerca volvulus). See, Cecil Textbook of Medicine, Wyngaarden et al. (ed.), W. B. Saunders Co., Philadelphia, Pa., 1992, pages 1971-2021.

Injectable composition: A pharmaceutically acceptable fluid composition comprising at least one active ingredient, e.g. a IGTP-family protein. The active ingredient is usually dissolved or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the GTPase proteins of this invention are conventional; appropriate formulations are well known in the art.

Isolated: An “isolated” biological component (such as a nucleic acid, peptide or protein) has been substantially separated, produced apart from, or purified away from other biological components in the cell of the organism in which the component naturally occurs, i.e., other chromosomal and extra-chromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Mammal: This term includes both human and non-human mammals. Similarly, the term “subject” includes both human and veterinary subjects.

Mimetic: This term includes both peptidomimetic and organomimetic compounds. Mimetics of the herein disclosed molecules are also hereby explicitly declared to be within the scope of the present invention, whereby the three-dimensional arrangement of the chemical constituents of such peptido- and organomimetics mimic the three-dimensional arrangement of the peptide backbone and component amino acid sidechains in the peptide, resulting in such peptido- and organomimetics of the peptides of this invention having substantial biological activity (e.g. anti-parasitic activity). For computer modeling applications, a pharmacophore is an idealized, three-dimensional definition of the structural requirements for biological activity. Peptido- and organomimetics can be designed to fit each pharmacophore with current computer modeling software (computer aided drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharnacology Munson (ed.) 1995, Ch. 102, for descriptions of techniques used in CADD. Also included within the scope of the invention are mimetics prepared using such techniques that produce IGTP-like proteins that retain the ability to modify immune responses, for instance anti-parasitic immune responses, and the use of such mimetics in the methods herein disclosed.

Proteins and peptides provided by the present invention can be chemically synthesized by any of a number of manual or automated methods of synthesis known in the art. Automated synthetic routines such as those available for use with automated peptide synthesizers are also intended to come within the scope of the present invention. Chemical derivatization, using the methods disclosed in this specification or other methods well known in the art, of naturally-occurring proteins or peptides or peptides purified from mixtures of protein degradation products, degraded by enzymatic or chemical means, are also within the scope of this invention, as are proteins or peptides made by molecular or genetic engineering means. Preferably, solid phase peptide synthesis (SPPS) is carried out on a 0.25 millimole (mmole) scale using an Applied Biosystems Model 431A Peptide Synthesizer and using 9-fluorenylmethyloxycarbonyl (Fmoc) amino-terminus protection, coupling with dicyclohexylcarbodiimide/hydroxybenzotriazole or 2-(1H-benzo-triazol-1-yl)-1,1,3,3-tetramethyluronium hexafluorophosphate/hydroxybenzotriazole (HBTU/HOBT), and using p-hydroxymethylphenoxymethylpolystyrene (HMP) or SasrinJ resin for carboxyl-terminus acids or Rink amide resin for carboxyl-terminus amides.

Fmoc-derivatized amino acids are prepared from the appropriate precursor amino acids by tritylation and triphenylmethanol in trifluoroacetic acid, followed by Fmoc derivatization as described by Atherton et al. (1989, Solid Phase Peptide Synthesis, IRL Press: Oxford).

SasrinJ resin-bound peptides are cleaved using a solution of 1% TFA in dichloromethane to yield the protected peptide. Where appropriate, protected peptide precursors are cyclized between the amino- and carboxyl-termini by reaction of the amino-terminal free amine and carboxyl-terminal free acid using diphenylphosphorylazide in nascent peptides wherein the amino acid sidechains are protected.

HMP or Rink amide resin-bound products are routinely cleaved and protected sidechain-containing cyclized peptides deprotected using a solution comprised of trifluoroacetic acid (TFA), optionally also comprising water, thioanisole, and ethanedithiol, in ratios of 100:5:5:2.5, for 0.5-3 hours at room temperature.

Crude peptides are purified by preparative high pressure liquid chromatography (HPLC) using a Waters Delta-Pak C18 column and gradient elution with 0.1% TFA in water modified with acetonitrile. After column elution, acetonitrile is evaporated from the eluted fractions, which are then lyophilized. The identity of each product so produced and purified is confirmed by fast atom bombardment mass spectroscopy (FABMS) or electrospray mass spectroscopy (ESMS).

Nucleic acid: A deoxyribonucleotide or nbonucleotide polymer in either single or double stranded form, and unless otherwise limited, encompasses known analogues of natural nucleotides that hybridize to nucleic acids in a manner similar to naturally occurring nucleotides.

Oligonucleotide: A linear polynucleotide sequence of up to about 200 nucleotide bases in length, for example a polynucleotide (such as DNA or RNA) which is at least 6 nucleotides, for example at least 15, 50, 100 or even 200 nucleotides long.

Therapeutically effective oligonucleotides and oligonucleotide analogs of the invention are additionally characterized by being sufficiently complementary to IGTP encoding nucleic acid sequences. As described herein, sufficient complementary means that the therapeutically effective oligonucleotide or oligonucleotide analog can specifically disrupt the expression of IGTP, and not significantly alter the expression of genes other than IGTP.

Oligopeptide: An oligopeptide is defined as a molecule of about 50 or fewer amino acid residues.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein encoding regions, in the same reading frame.

ORF: A series of nucleotide triplets (codons) coding for amino acids without any internal termination codons. These sequences are usually translatable into a peptide.

Ortholog: Genes of similar function, but occurring in different species. Orthologs need not be but are often also homologs.

Parasite: Living entities that dwell in or on other creatures (the hosts) during some part of their life cycle, drawing nourishment from the host. As used herein, the term parasite includes protozoan and helminth infectious agents.

Parenteral: Administered outside of the intestine, e.g. not via the alimentary tract. Generally, parenteral formulations are those that will be administered through any possible mode except ingestion. This term especially refers to injections, whether administered intravenously, intrathecally, intramuscularly, intraperitoneally, or subcutaneously, and various surface applications including intranasal, intradermal, and topical application, for instance.

PCR: Describes a technique in which cycles of denaturation, annealing with primer, and then extension with DNA polymerase are used to amplify the number of copies of a target DNA sequence.

Peptide Nucleic Acid (PNA): An oligonucleotide analog with a backbone comprised of monomers coupled by amide (peptide) bonds, such as amino acid monomers joined by peptide bonds.

Pharmaceutical agent or drug: A chemical compound or composition capable of inducing a desired therapeutic or prophylactic effect when properly administered to a subject

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the IGTP-family proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g. powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Primers: Short nucleic acids, for instance DNA oligonucleotides 10 nucleotides or more in length, which are annealed to a complementary target DNA strand by nucleic acid hybridization to form a hybrid between the primer and the target DNA strand, then extended along the target DNA strand by a DNA polymerase enzyme. Primer pairs can be used for amplification of a nucleic acid sequence, e.g., by the polymerase chain reaction (PCR) or other nucleic acid amplification methods known in the art. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Probes and primers as used in the present invention may comprise at least 10 nucleotides of the nucleic acid sequences. In order to enhance specificity, longer probes and primers may also be employed, such as probes and primers that comprise at least 15, 20, 30, 40, 50, 60, 70, 80, 90 or 100 or more consecutive nucleotides of an IFN-γ inducible GTPase. Methods for preparing and using probes and primers are described in the references, for example Sambrook et al. Molecular Cloning: A Laboratory Manual, Cold Spring Harbor, N.Y., 1989; Ausubel et al. Current Protocols in Molecular Biology, Greene Publ. Assoc. & Wiley-Intersciences, 1987; Innis et al. PCR Protocols, A Guide to Methods and Applications, Innis et al. (Eds.), Academic Press, San Diego, Calif., 1990. PCR primer pairs can be derived from a known sequence, for example, by using computer programs intended for that purpose such as Primer (Version 0.5, 1991, Whitehead Institute for Biomedical Research, Cambridge, Mass.).

When referring to a probe or primer, the term “specific for” (a target sequence) indicates that the probe or primer hybridizes under stringent conditions substantially only to the target sequence in a given sample comprising the target sequence.

Probe: An isolated nucleic acid attached to a detectable label or reporter molecule. Typical labels include radioactive isotopes, ligands, chemiluminescent agents, and enzymes.

Promoter: A promoter is an array of nucleic acid control sequences that direct transcription of a nucleic acid. A promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

Protein purification: The polypeptides of the present invention can be purified by any of the means known in the art. See, e.g., Guide to Protein Purification, ed. Deutscher, Meth. Enzymol. 185, Academic Press, San Diego, 1990; and Scopes, Protein Purification: Principles and Practice, Springer Verlag, New York, 1982.

Purified: The term purified does not require absolute purity; rather, it is intended as a relative term. Thus, for example, a purified IGTP protein preparation is one in which the IGTP protein is more enriched than the protein is in its generative environment, for instance within a cell or in a biochemical reaction chamber. A preparation of IGTP-family protein may be purified such that the desired protein represents at least 50% of the total protein content of the preparation.

Recombinant: A recombinant nucleic acid molecule is one that his a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques.

Similarly, a recombinant protein is one encoded for by a recombinant nucleic acid molecule.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs of an IGTP-family protein, and the corresponding cDNA or gene sequence, will possess a relatively high degree of sequence identity when aligned using standard methods. This homology usually will be more significant when the proteins or genes or cDNAs are derived from species that are more closely related (e.g., human and chimpanzee sequences), compared to species more distantly related (e.g., human and C. elegans sequences).

Methods of alignment of sequences for comparison are well known in the art. Various programs and alignment algorithms are described in: Smith & Waterman Adv. Appl. Math., 2: 482, 1981; Needleman & Wunsch J. Mol. Biol., 48: 443, 1970; Pearson & Lipman Proc. Natl. Acad. Sci. USA, 85: 2444, 1988; Higgins & Sharp Gene, 73: 237-244, 1988; Higgins & Sharp CABIOS, 5: 151-153, 1989; Corpet et al. Nuc. Acids Res., 16, 10881-90, 1988; Huang et al. Computer Appls. in the Biosciences, 8, 155-65, 1992; and Pearson et al. Meth. Mol. Bio., 24, 307-31, 1994. Altschul et al (J. Mol. Biol., 215:403-410, 1990) presents a detailed consideration of sequence alignment methods and homology calculations.

The NCBI Basic Local Alignment Search Tool (BLAST) (Altschul et al. J. Mol. Biol., 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI BLAST Internet site (http://www.ncbi.nlm.nih.gov/BLAST/). A description of how to determine sequence identity using this program is available at the NCBI BLAST help site, also on the Internet (at http://www.ncbi.nlm.nih.gov/BLAST/blast_help.html).

Homologs of IGTP-family proteins, for instance homologs of murine IGTP, typically possess at least 60% sequence identity counted over full-length alignment with the amino acid sequence of murine IGTP using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 70%, at least 75%, at least 80%, at least 90%, at least 95%, at least 98%, or at least 99% sequence identity. When less than the entire sequence is being compared for sequence identity, homologs will typically possess at least 75% sequence identity over short windows of 10-20 amino acids, and may possess sequence identities of at least 85% or at least 90% or at least 95% depending on their similarity to the reference sequence. Methods for determining sequence identity over such short windows are described at the NCBI BLAST Internet help page (http://www.ncbi.nlm.nih.gov/BLAST/blast_FAQs.html).

One of ordinary skill in the art will appreciate that these sequence identity ranges are provided for guidance only; it is entirely possible that strongly significant homologs could be obtained that fall outside of the ranges provided. The present invention provides not only the peptide homologs that are described above, but also nucleic acid molecules that encode such homologs.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tm is the temperature (under defined ionic strength and pH) at which 50% of the target sequence remains hybridized to a perfectly matched probe or complementary strand. Conditions for nucleic acid hybridization and calculation of stringencies can be found in Sambrook et al. (Molecular Cloning: A Laboratory Manual, CSHL, New York, 1989) and Tijssen (Laboratory Techniques in Biociemistry and Molecular Biology—Hybridization with Nucleic Acid Probes Part I, Chapter 2, Elsevier, N.Y., 1993). Nucleic acid molecules that hybridize under stringent conditions to a murine IGTP encoding sequence will typically hybridize to a probe based on either an entire murine IGTP encoding sequence or selected portions of the encoding sequence under wash conditions of 2×SSC at 50° C.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid molecules that all encode substantially the same protein.

Specific binding agent: An agent that binds substantially only to a defined target. Thus a “IGTP-specific binding agent” binds substantially only the IGTP protein. As used herein, the term “IGTP-specific binding agent” includes anti-IGTP antibodies and other agents that bind substantially only to a IGTP protein.

Antibodies to IGTP-family proteins may be produced using standard procedures described in a number of texts, including Harlow and Lane (Using Antibodies, A Laboratory Manual, CSHL, New York, 1999, ISBN 0-87969-544-7). The determination that a particular agent binds substantially only to IGTP protein may readily be made by using or adapting routine procedures. One suitable in vitro assay makes use of the Western blotting procedure (described in many standard texts, including Harlow and Lane, 1999). Western blotting may be used to determine that a given protein binding agent, such as an anti-IGTP monoclonal antibody, binds substantially only to the IGTP protein. Antibodies to IGTP are well known in the art.

Shorter fragments of antibodies, which still retain specific binding capability, can also serve as specific binding agents. For instance, Fabs, Fvs, and single-chain Fvs (SCFvs) that bind to IGTP would be IGTP-specific binding agents. These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. Methods of making these fragments are routine.

Target sequence: “Target sequence” is a portion of ssDNA, dsDNA or RNA that, upon hybridization to an therapeutically effective oligonucleotide or oligonucleotide analog, results in the inhibition of IGTP expression. Either an antisense or a sense molecule can be used to target a portion of dsDNA, since both will interfere with the expression of that portion of the dsDNA. The antisense molecule can bind to the plus strand, and the sense molecule can bind to the minus strand. Thus, target sequences can be ssDNA, dsDNA, and RNA.

Therapeutically effective amount of a IGTP-family protein: A quantity of IGTP-family protein sufficient to achieve a desired effect in a subject, for instance an amount sufficient as a treatment, a prophylactic, or a replacement agent For instance, this can be the amount necessary to measurably inhibit infectivity of a biological infectious agent (e.g., a virus, bacteria, protozoa, yeast or other fungus).

An effective amount of IGTP-family protein (e.g., IGTP) may be administered in a single dose, or in several doses, for example daily, during a course of treatment. However, the effective amount of IGTP-family protein will be dependent on the IGTP-family protein applied, the subject being treated, the severity and type of the affliction, and the manner of administration of the IGTP-family protein. For example, a therapeutically effective amount of IGTP-family protein can vary from about 0.01 mg/kg body weight to about 1 g/kg body weight.

The IGTP-family proteins disclosed in the present invention have equal application in medical and veterinary settings. Therefore, the general term “subject being treated” is understood to include all animals (e.g., humans, apes, mice, dogs, cats, horses, and cows) that are or may be infected with a protozoan parasite or other disease-causing microorganism (e.g., bacteria, virus, yeasts) that is susceptible to immune responses such as immune responses involving IFN(.

Transformed: A transformed cell is a cell into which has been introduced a nucleic acid molecule by molecular biology techniques. A cell is “transformed” by a nucleic acid when the DNA becomes stably replicated by the cell, either by incorporation of the nucleic acid into the cellular genome, or by episomal replication. As used herein, the term transformation encompasses all techniques by which a nucleic acid molecule might be introduced into such a cell, including transfection/transduction with viral vectors, transformation with plasmid vectors, and introduction of naked DNA by electroporation, lipofection, and particle gun acceleration.

For instance, a virus or vector “transduces” a cell when it transfers nucleic acid into the cell.

Variant oligonucleotides: A “variant oligonucleotide” is an oligomer having one or more base substitutions (i.e. naturally occurring bases such as A, T, C, G, or U, or synthetic bases such as those described below), one or more base deletions, and/or one or more base insertions, so long as the oligomer substantially retains the activity of the original oligonucleotide, or has sufficient complementarity to a target sequence.

A variant oligonucleotide is additionally characterized by its ability to hybridize to the target sequence, under stringency conditions that are sufficient to disrupt the expression of an IGTP-family protein (such as IGTP, LRG-47, IRG-47, and so forth).

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art A vector can transduce, transform or infect a cell, thereby causing the cell to express nucleic acids and/or proteins other than those that are native to the cell. A vector optionally includes materials to aid in achieving entry of the nucleic acid into the cell, such as a viral particle, liposome, protein coating or the like.

II. Characterization of IGTP-family Proteins

The studies presented here indicate IGTP-family proteins are essential components of a novel IFNγ-regulated pathway. This pathway mediates host resistance to pathogens, surprisingly including both bacteria such as L. monocytogenes and protozoa such as T. gondii. Even more remarkably, individual IGTP-family proteins contribute differentially to different resistance of a host/subject to specific microbial pathogens.

The prototype of the IFNγ-induced GTPase family, IGTP, has been shown to be essential for IFNγ-induced immune defense against acute infections of the protozoan parasite Toxoplasma gondii, while it is not required for defense against the bacterium Listeria monocytogenes or against murine cytomegalovirus (MCMV). In contrast to IGTP-deficient mice, LRG-47-deficient mice displayed decreased resistance to both acute T. gondii and L. inonocytogenes infections, while maintaining normal resistance to MCMV. Conversely, IRG-47-deficient mice displayed only partially decreased resistance to T. gondii during the chronic phase of infection, and completely normal resistance to L. monocytogenes and MCMV. Thus, the members of the IGTP family of IFNγ-induced genes have vital, but distinct roles in mediating host defense against infectious agents.

Each specific IGTP-family protein may be involved in host resistance to other pathogens, but this effect may be masked in the current experiments due to compensatory up-regulation or substitution of IGTP-family member proteins with potentially overlapping function, for instance TGTP/Mg21 (Carlow et al., J. Inmunol., 154:1724-1734, 1995; Lafuse et al., J. Leukoc. Biol., 57:477483, 1995), IIGP (Boehm et al., J. Immunol., 161:6715-6723, 1998), or GTPI (Boehm et al., J. Immunol., 161:6715-6723, 1998), for functional IGTP, LRG-47, and/or IRG-47. It is possible that these proteins may operate within the same IFNγ-regulated pathway, or alternatively that they may regulate different anti-microbial pathways that are all controlled or influenced by IFNγ, possibly in combination with other cytokines.

This disclosure presents methods of modifying the immune response in a subject, particularly anti-bacterial and/or anti-parasite immunity, by modifying the activity of an IGTP-family protein or a related protein in the subject Such modification can be through an increase or decrease in the level of IGTP-family protein expression, for instance by expression of a recombinant IGTP-family protein-encoding nucleic acid, or by administration to the subject of an IGTP-family protein, or a fragment, variant, analog, derivative or mimetic thereof that maintains immune response modifying activity. These methods can be used to treat subjects that are infected with or at risk for infection with an infectious biological agent, for instance a virus, a bacterium or a parasite.

In certain embodiments presented herein, the immune response treated by modifying IGTP-family activity in a subject is a Th1 immune response. Such a response can be directed against an infectious agent (e.g., a virus, a bacterium or a parasite), or can be an auto-immune disease condition. Examples of parasites include Toxoplasma sp., Plasmodium sp., Tryapanosoma sp., Leishmania sp., Cryptosporidium sp., Giardia sp., Entamoeba sp., Trichomonas sp., Diphyllobotlriunm latum, Echinococcosis sp., Schistosoma sp., Wuchereria bancrofti, Brugia malayi and Onchocerca volvulus. Examples of auto-immune disease conditions include those that are mediated by over- or under-active Th1 immune activity, such as irritable bowel disease (IBD), rheumatoid arthritis, auto-immune diabetes mellitus, lupus erythematosis, sarcoidosis, multiple sclerosis, chronic delayed type hypersensitivity (DTH), and auto-immune encephalomyelitis. Examples of bacteria include Streptococcus sp., Haemophilus influenzae, Klebsiella sp., Escherichia sp., Legionella sp., Mycoplasma sp., Pneunocystis carinii, Listeria, Corynebacterium sp., Staphylococcus sp., Serratia, Pseudomonas, Shigella, Vibrio, Heinophilus sp., Yersinia, Enterobacter, Mycobacteria, Chlamydiae, and rickettsiae organisms, for instance. Examples of viruses include viruses of the following families: Poxviridae, Herpesviridae, Adenoviridiae, Papovaviridae, Hepadnaviridae, Parvoviridae, Reviridae, Togoviridae, Flaviviridae, Coronaviridae, Paramyxoviridae, Rhabdoviridae, Bunyaviridae, Arenaviridae, Retroviridae, Picornaviridae, and Caliciviridae.

Depending on the IGTP-family protein or encoding nucleic acid used, in certain aspects of this invention an immune response is either enhanced or inhibited.

In a further embodiment, IGTP expression is altered by expressing a recombinant genetic construct that contains a promoter operably linked to a nucleic acid molecule, wherein the nucleic acid molecule includes at least 10 consecutive nucleotides of a nucleotide sequence that encodes IGTP, and expression of the nucleic acid molecule changes expression of IGTP. One appropriate nucleotide sequence that can be used for this embodiment is GenBank Accession Number U53219. The construct of this embodiment can either increase or decrease IGTP expression, depending on the components used in its construction. Likewise, encompassed herein are embodiments wherein LRG-47 (U19119) or IRG-47 (M63630) expression is altered.

Certain other embodiments include methods for inhibiting replication or infectivity of an infectious agent in a subject, or treating or preventing infection of the subject by the infectious agent. Such methods involve administering to the subject an amount of an IGTP-family protein or encoding sequence, or a fragment, variant, analog or mimetic thereof, sufficient to inhibit infectious agent replication or infectivity. An example of an infectious agent against which immunity can be enhanced in this manner is a parasite, such as a helminth or protozoan parasite, or more particularly protozoa such as T. gondii or other parasites listed herein. Other infectious agents against which immunity can be enhanced are bacteria, such as those listed herein, for instance L. monocytogenes. Still other infectious agents against which immunity can be enhanced are viruses, such as those listed herein.

These methods and molecules can be used to enhance immunogenicity of an antigen (such as a protozoan antigen) that evokes an immune response. The molecules can also be used as adjuvants, to stimulate for instance, Th1-mediated immune responses in target cells.

A still further embodiment is a method for detecting susceptibility of a subject to parasitic, bacterial, or viral infection. In such a method, abnormal an IGTP-family protein, abnormal IGTP-family protein expression, or abnormal IGTP-family encoding nucleic acid is detected in the subject Various methods can be used to detect these abnormal molecules, such as binding a complementary oligonucleotide to an IGTP-family member encoding sequence or detecting or quantitating an IGTP-family protein using a protein-specific protein binding agent (e.g. an antibody).

Also disclosed are kits for use with these various methods. Depending on the method, such kits will include one or more IGTP-family protein-specific binding agents (e.g., an IGTP-specific, IRG-47-specific, or LRG-47-specific antibody) or one or more IGTP-family member encoding nucleic acid-specific binding agents (e.g., a probe or primer that includes at least 10 nucleotides from the nucleic acid sequence of GenBank Accession Number U53219, U19119, or M63630).

Transgenic non-human animals in which expression of an IGTP-family gene has been altered are also encompassed by the present disclosure. In such animals, an IGTP-family gene may be over- or under-expressed relative to expression prior to such gene expression alteration. These transgenic animals can be used to study the immune response, as well as in methods of screening for anti-microbial (e.g., anti-viral, anti-bacterial and/or anti-protozoan) compounds, for instance by administering a candidate compound to the transgenic animal. Appropriate candidate compounds include anti-microbial drugs, for instance analogs of recognized anti-viral, anti-bacterial or anti-protozoan drugs. IGTP-family protein deficient animals, for example, can be used to detect anti-microbial pharmaceutical activity in the absence of an IGTP-family member-mediated or influenced immune response. Alternatively, IGTP-family-member over-expressing animals can be used to study an immune response following exposure of the transgenic animal to an immunogen, such as an antigen of interest.

Pharmaceutical compositions encompassed by the current invention may also include a pharmaceutically acceptable vehicle or carrier, a therapeutically effective amount of at least one anti-protozoan compound, and a therapeutically effective amount of at least one IGTP-family protein, or a fragment, variant, analog, derivative, or mimetic thereof, or a nucleic acid that encodes the IGTP-family protein, fragment, variant, analog, derivative, or mimetic thereof. The anti-protozoan compound that is included in such compositions may be, for instance, a protozoan antigen that evokes an immunogenic response to the protozoan, a bacterial antigen that evokes an immunogenic response to the bacterium, or an anti-microbial pharmaceutical compound (e.g., one that is effective in treating or preventing a Toxoplasma infection, a Listeria infection, or another microbial infection).

A. IGTP Characterization

### Materials and Methods

### IGTP Gene Targeting

An IGTP targeting vector (FIG. 1A) was constructed from a 6 kb XbaI IGTP gene fragment, from which 0.9 kb of the IGTP gene corresponding to intronic sequence and codons 13 through 240 (Taylor et al., J. Biol. Chem., 271:20399-20405, 1996) were deleted and replaced with pGKneoBpA. In the vector, these sequences were flanked by pGKtkBpA (Porter and Sande, New Engl. J. Med., 327:1643-1648, 1992). The targeting vector was electroporated into CJ7 ES cells (Porter and Sande, New Engl. J. Med., 327:1643-1648, 1992), and homologous recombinants were selected by Southern blotting of BamHI digested DNA with a 5′ external GTP probe (a 0.18 kb BamHI-NcoI fragment of the IGTP cDNA). Using these cells and established procedures, IGTP-deficient mice were generated (Porter and Sande, New Engl. J. Med., 327:1643-1648, 1992). Two lines of IGTP-deficient mice were established from separate targeted ES cell lines, and the behavior of the two mouse lines was indistinguishable in subsequent studies. All experiments were performed using 1-4 month old mice on a C57BL/6×129Sv genetic background. The mice were maintained in a specific pathogen-free facility.

### Protein and RNA Analyses

For western blot analysis, protein lysates were prepared from cells or tissues, separated by 10% SDS-PAGE, and blotted with anti-IGTP antibodies, as described previously (Taylor et al., J. Biol. Chem., 271:20399-20405, 1996). For northern blot analysis, 15 μg total RNA samples were separated on 1.2% agarose/formaldehyde gels, and blotted with mouse IGTP and human GAPDH cDNA probes, as described previously (Taylor et al., J. Biol. Chem., 271:20399-20405, 1996).

### Toxoplasma gondii Infection

Mice were injected, i.p., with 0.5 mL PBS containing 20 cysts of the avirulent ME49 strain of Toxoplasma gondii, that had been prepared from the brains of infected C57 BL/6 mice. The mice were monitored daily.

For ex vivo cytokine analysis, single-cell spleen cell and peritoneal exudate cell cultures were prepared from infected mice, and red cells were removed from the spleen cells using ACK lysing buffer (Bio Whittaker, Walkersville, Md.). The spleen cells and PECs were then cultured in 96 well plates, at 8×10⁵ and 4×10⁵ cells per well respectively, in 200 μl RPMI medium (Life Technologies, Gaithersberg, Md.) supplemented with 10% (v/v) fetal bovine serum (Life Technologies). When indicated, cell cultures were stimulated with 10 mg/mL plate-bound anti-CD3 (Pharmingen, San Diego, Calif.) or 10 mg/mL STAg (soluble tachyzoite antigen), which had been prepared from sonicated RH parasites as previously described (Grunvald et al., Infect. Immun., 64:2010-2018, 1996). Conditioned media were collected 72 hours later for determination of IFNγ and IL-12 p40 levels using sandwich ELISA as described previously (Scharton-Kersten, et al., J. Immunol., 157:4045-4054, 1996).

Sera were prepared from blood that was collected at the time of sacrifice, allowed to clot, and then centrifuged at 6000 rpm for 10 minutes.

### Listeria monocytogenes Infection

The mice were inoculated with different doses i.p. of L. monocytogenes EGD strain (provided by Dr. K Elkins, U.S. Food and Drug Administration). Health and survival of the mice were monitored daily for at least 14 days. For experiments involving the measurement of serum cytokine levels, bacterial loads in the spleen and liver, or IGTP expression levels, the mice were inoculated with 1000 bacteria i.p., and the sera and relevant tissues were isolated three days later. Bacterial counts were determined by excising spleens and livers sterilely, homogenizing portions of the organs in PBS, and plating serial dilutions of the homogenate on LB agar plates. Colony counts were assessed the following day, and the total bacterial load per organ was calculated.

### Viral Infection

MCMV (Orange et al., J. Exp. Med., 182:1045-1056, 1995) and Ebola virus infection (Bray et al., J. Infect. Dis., 178:651-661, 1998), and the subsequent hepatic analysis, and cytokine ELISA were performed as described in detail in the cited references.

### Results

To target the IGTP gene in order to knock it out, a specific targeting vector was constructed in which the majority of the IGTP protein coding sequence was replaced by a neomycin resistance gene (FIG. 1A). Using this vector, IGTP-deficient mice were generated. These mice produced no detectable IGTP protein in all tested tissues, including thymus (FIG. 1B). These mice displayed no obvious physical or behavioral abnormalities; they were fertile; and the targeted IGTP allele segregated with near Mendelian ratios. In addition, complete histological analysis of the major organs indicated nothing remarkable, and FACS analysis revealed no changes in immune cell development, as assessed by examining T cell (CD3+, CD4+, CD8+, CD25+), B cell (B220+), NK cell (DX5+), granulocyte (Mac1+, 8C5+), macrophage (Mac1+, 8C5−, DX5−), and erythroid (TER119+) cell markers in the thymus, spleen, bone marrow, and lymph nodes.

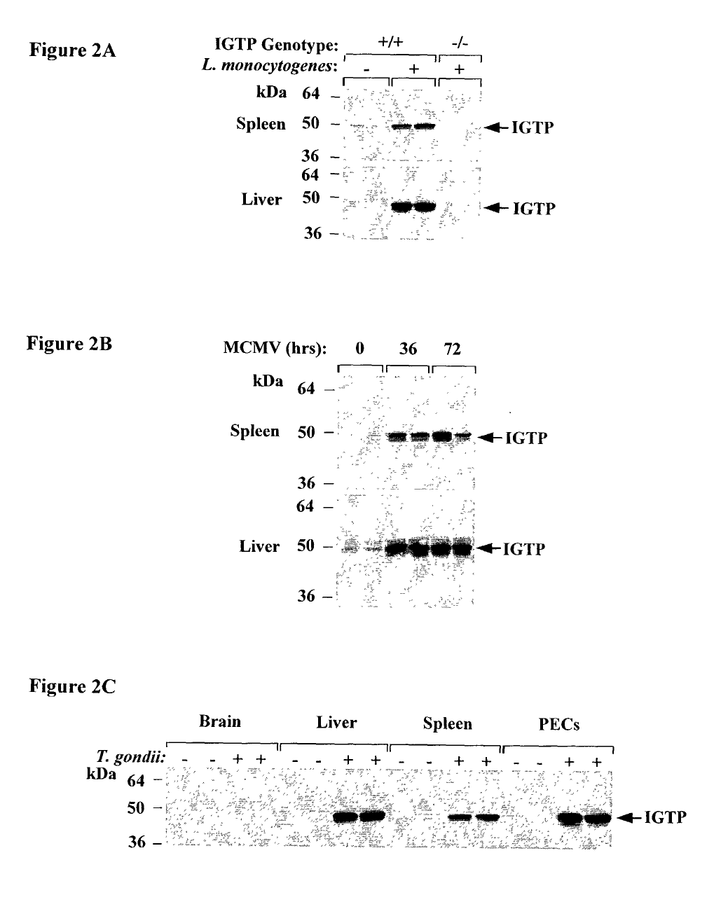

To examine host defense against pathogens in the IGTP-deficient mice, they were challenged with several infectious agents including the protozoan parasite Toxoplasma gondii, the bacterium Listeria monocytogenes, and murine cytomegalovirus (MCMV). Resistance to each of these pathogens is crucially dependent on host production of IFNγ; mice lacking IFNγ or the IFNγ receptor demonstrate markedly increased susceptibility to each of them (Huang et al., Science, 259:1742-1745, 1993; Scharton-Kersten, et al., J. Immunol., 157:4045-4054, 1996; Orange et al., J. Exp. Med., 182:1045-1056, 1995, Gazzinelli et al., J. Immunol., 153:2533-2543, 1994; Pomeroy et al., J. Lab. Clin. Med., 132:124-133, 1998). In the present studies, infection of wild-type mice with any of the three pathogens dramatically increased IGTP expression in liver and spleen (FIG. 2). However, despite the uniformly increased IGTP levels seen in wild-type mice, IGTP-deficient mice displayed severely compromised defense specifically against T. gondii, but not against the bacterium or virus.

### Bacterial Infection

In the L. monocytogenes bacterial studies, all IGTP-deficient and wild-type mice inoculated with 800 or fewer bacteria survived the challenge, while all IFNγ-deficient mice receiving 80 bacteria uniformly succumbed within 4-5 days (FIG. 3A). At higher innocula, the IGTP-deficient and wild-type mice showed similar mortality rates (FIG. 3A). Furthermore, splenic and hepatic bacterial loads three days following inoculation with 1000 bacteria were about equivalent in IGTP-deficient and wild-type mice, but were increased 3-4 logs in IFNγ receptor-deficient mice (FIG. 3B).

### Viral Infection

In the MCMV studies, IGTP-deficient and wild-type mice had comparable hepatic viral loads (FIG. 3C), as well as similar incidences of focal hepatic necrosis (not shown), at both 36 and 72 h following MCMV inoculation. Conversely, it has been shown previously that IFNγ-deficient mice have marked increases in both hepatic viral titers and hepatic necrosis after MCMV infection (Orange et al., J. Exp. Med., 182:1045-1056, 1995; Pomeroy et al., J. Lab. Clin. Med., 132:124-133, 1998). In addition, the IGTP-deficient mice displayed undiminished cytotoxic T lymphocyte activity and NK cell cytolytic activity. IGTP-deficient mice were also challenged with Ebola virus, but they showed neither altered susceptibility to Ebola, nor a defect in their ability to be immunized against it.

### Parasitic Protozoa Infection

In striking contrast to the bacterial and viral studies, the IGTP-deficient mice showed a complete inability to restrict acute T. gondii infection, which was used as a model for parasitic protozoan infection. All IGTP-deficient mice inoculated i.p. with 20 cysts of the parasite died within 8-12 days, while wild-type mice survived the infection (FIG. 3D). This pronounced susceptibility to the parasite mimicked that previously reported for IFNγ-deficient mice, which died within the same time frame (Scharton-Kersten, et al., J. Immunol., 157:4045-4054, 1996), and it suggested that IGTP was essential for IFNγ-dependent host resistance to T. gondii.

To directly assess the ability of the mice to restrict parasite replication, IGTP-deficient and wild-type mice were inoculated with T. gondii and measured parasitic loads at 5 days post-infection. By microscopic examination, 19+/−5 (sem) % of peritoneal exudate cells (PECs) from IGTP-deficient mice contained parasites, compared to 0.05+/−0.05% of those from wild-type mice, indicating that immune defense was not sufficient to prevent spread of the parasite.

IL-12 p40 and IFNγ levels were also measured at 5 days post-infection; increased production of these cytokines is absolutely required to restrict T. gondii infection (Scharton-Kersten, et al., J. Immunol., 157:40454054, 1996; Gazzinelli et al., J. Immunol., 153:2533-2543, 1994; Gazzinelli et al., Proc. Natl. Acad. Sci., 90:6115-6119, 1993). In sera, both IL-12 and IFNγ levels were slightly elevated in infected IGTP-deficient mice, compared to infected wild-type mice (FIGS. 4A & 4D). From splenocytes and PECs isolated from infected IGTP-deficient mice and cultured in vitro, there was also slightly increased IFNγ and IL-12 production; these levels increased when the cells were stimulated with anti-CD3 or a Toxoplasma antigen mixture (STAg), to levels comparable to those secreted by infected wild-type cells (FIGS. 4B, 4C, 4E & 4F). Therefore, the IGTP-deficient mice responded to T. gondii infection with a robust IFNγ response, implying that their defect in controlling T. gondii was distal to IFNγ. The slightly increased level of IL-12 and IFNγ was probably only a result of the persistent infection.

In the IGTP-deficient mice, production of nitric oxide (NO), an important effector of macrophage based killing, was also examined. Studies with mice deficient in inducible nitric oxide synthase (iNOS), the enzyme which generates NO, have shown that NO is essential for resistance to chronic T. gondii infections, but not acute infections (Scharton-Kersten et al., J. Exp. Med., 185:1261-1273, 1997). IGTP-deficient mice showed large increases in hepatic iNOS mRNA levels 8 days after T. gondii infection; the iNOS mRNA levels in infected wild-type mice were also increased, but not to the same extent (FIG. 5). However, in infected IFNγ receptor-deficient mice, iNOS levels were increased only slightly (FIG. 5). Higher iNOS levels in the IGTP-deficient mice, relative to those in wild-type mice, may have been due to the uncontrolled infection. However, the retention of a marked increase in iNOS levels in IGTP-deficient mice following infection suggested that their lack of host resistance to T. gondii was not simply a result of decreased NO production.

In the context of host resistance, IGTP could function by regulating immune cell function, or alternatively, by providing an anti-microbial activity in all cells including nonimmune cells. As to the former, no evidence has been found to indicate that IGTP regulates any specific immune cell activity. For instance, in vivo production of IL-12 p40, IFNγ, and NO by IGTP-deficient mice was not decreased following acute T. gondii infection; cytotoxic T lymphocyte and NK cell killing in IGTP-deficient cells was not reduced; and IGTP-deficient splenocytes showed undiminished proliferative responses to T cell and B cell mitogens. Furthermore, the observation that IGTP-deficient mice display normal resistance to L. monocytogenes argues that IGTP deficiency does not result in generalized immune deficiency or dysregulation.

The data therefore indicate that IGTP provides a generalized effect against a protozoan parasite, T. gondii, in many types of cells, not only in immune cells. IFNγ-induced elements are required in both immune cells and nonimmune cells for normal host resistance to protozoan parasites such as T. gondii (Yap and Sher et al., J. Ex. Med., 189:1083-1091, 1999). This is in contrast to resistance to L. monocytogenes, in which IFNγ action is only required in immune cells (Yap and Sher et al., J. Ex. Med, 189:1083-1091, 1999). IGTP is expressed in immune and nonimmune cells, so that it is capable of providing critical elements of host resistance to T. gondii within both cell types. Established T. gondii is somewhat sequestered from the endocytic and exocytic machinery of the host cells, within a parasitophorous vacuole, where parasite growth occurs. Not meaning to be bound by any particular explanation, it is possible that, through regulation of vesicular movement to the parasitophorous vacuole, IGTP influences or controls parasite clearance. This mechanism may involve the targeted transport of IFNγ-induced toxic mediators to the vacuole, and thereby to the parasite.

With the provision herein of a native function of the IGTP protein, the usefulness of this and closely related molecules (including fragments, variants, analogs, derivatives and mimetics of the IGTP protein that maintain immune response modifying activity, and nucleic acid sequences that encode such) is now exploitable. IGTP molecules are useful in the prevention or reduction of infection in animals. In addition, these molecules are useful for treatment of auto-immunune disease conditions in animals, which are influenced by Th1-mediated inununological responses.

B. LRG-47 and IRG-47 Characterization

Gene targeting was used to determine whether other members of the IGTP protein family have important roles in host defense, similarly to IGTP itself. Two strains of mice were created that lacked expression of representatives of the two subgroups of the IGTP protein family: LRG-47 and IRG-47. The resulting phenotypes of the LRG-47 and IRG-47-deficient mice demonstrate that the two genes are, in fact, critical mediators of host resistance. Furthermore, they suggest that each gene in the IGTP protein family evolved to fill a distinct niche in an IFNγ-coordinated immune program to clear invading pathogens.

### Methods

### LRG-47 and IRG-47 Gene Targeting

To construct an LRG-47 targeting vector, the gene was cloned from a 129SvJ mouse library (Stratagene, La Jolla, Calif.) using the LRG-47 cDNA as probe. Contiguous 5 kb and 3 kb XbaI fragments were cloned that contained the entire LRG-47 protein coding region (GenBank U19119), as well as a single intron within the coding region. In-the targeting vector, a 0.95 kb SpeI-XbaI portion of the 5 kb fragment, including 0.7 kb of the protein coding region, was deleted and replaced with pGKneoBpA that served as a positive selective marker. These sequences were then flanked by pGKtkBpA (Bonin et al., Methods in Mol Biol., in press), a negative selective marker.

To construct an IRG-47 targeting vector, the gene was cloned from a 129SvJ library using the IRG-47 cDNA as a probe. Two fragments of the gene were cloned: a 3.0 kb SacI fragment that contained the 5′ portion of the protein coding region (GenBank M63630) and upstream sequences, and a 5.5 kb XbaI fragment that contained the 3′ untranslated region (GenBank M63630) and downstream seqences. The targeting vector was created by separating a 2 kb HindIII-NcoI portion of the 3.0 kb SacI fragment, and the entire 5.5 kb XbaI fragment, with a pGKneoBpA (Bonin et al., Methods in Mol Biol., in press), which in effect deleted the entire protein coding region of the gene. These sequences were then flanked with pGKtkBpA (Bonin et al., Methods in Mol Biol., in press).

The targeting vectors were electroporated into CJ7 embryonic stem cells (Bonin et al., Methods in Mol Biol., in press.), and homologous recombinants were selected by Southern blotting of EcoRI-resticted DNA with an LRG-47 probe (a 0.5 kb BglII of the LRG-47 cDNA fragment) or an IRG-47 probe (a 0.5 kb SacI-HindIII fragment of the 3.0 kb SacI IRG-47 genomic clone). Using the targeted cells and established procedures, IGTP-deficient mice were generated on a C57B1/6×129SvJ genetic background. All experiments were performed with 1-4 month old mice, and the mice were housed in a specific pathogen-free facility.

### Protein and RNA Analyses

For western blotting, protein lysates were isolated from cells or tissues, separated by 10% SDS-PAGE, and blotted as described previously (Taylor et al., J. Biol. Chem., 271:20399-20405, 1996). Rabbit polyclonal anti-LRG-47 antisera recognized the internal LRG-47 peptide sequence corresponding to amino acid residues 35-50 of GenBank Accession Number U19119 (Sorace et al., J. Leukoc. Biol., 58:477-484, 1995), except that the glutamine corresponding to residue 36 was an asparagine in the peptide. Rabbit polyclonal antisera recognized the C-terminal IRG-47 sequence corresponding to amino acid residues 405-420 of GenBank Accession Number M63630 (Gilly & Wall, J. Immunol., 148:3275-3281, 1992), except that the glutamic acid corresponding to residue 415 was an aspartic acid in the peptide.

For northern blot analysis, 15 μg total RNA samples were separated on 1.2% agarose/formaldehyde gels and blotted with labeled probes as described previously (Taylor et al., J. Biol. Chem., 271:20399-20405, 1996). The probes included a human glyceraldehyde phosphate dehydrogenase probe isolated as a 1.2 kb fragment of pHcGAP (Beutler et al., J. Exp. Med., 164:1791-1796, 1986), a mouse IGTP 3′ untranslated region probe isolated as a 0.28 kb EcoRI fragment (Taylor et al., J. Biol. Chem., 271:20399-20405, 1996), a mouse LRG-47 cDNA probe isolated as a 1.4 kb KpnI fragment of the LRG-47 cDNA (GenBank U19119), and a mouse IRG-47 3′ untranslated region probe corresponding to bases 1374 to 1625 (GenBank M63630) that were isolated using the polymerase chain reaction.

### Flow Cytometry

Spleen tissues were teased with a 1 cc tuberculin syringe plunger (Becton Dickinson, Franklin Lakes, N.J.) to achieve a single cell suspension. Splenocytes were then washed in RPMI 1640 medium with L-glutamine (Life Technologies) supplemented with 10% FCS (Hyclone, Logan, Uath), 5.5×10⁻⁵ M 2-ME, and 10 μg/ml gentamicin (BioWhittaker, Walkersville, Md.), and counted on a Coulter Counter (Coulter Electronics, Hialeah, Fla.). 10⁶ cells were incubated (30 minutes, 4° C.) with saturating amounts of antibody. MAbs used for immunofluorescent staining included rat anti-mouse CD3 (Sigma, St. Louis, Mo.), rat anti-mouse CD4 (Caltag, Burlingame, Calif.), anti-mouse B220, anti-mouse F480, and anti-mouse NK (BD Pharmingen, San Diego, Calif.). After labeling, the cells were washed 2 times with 3 ml PBS wash (1×PBS, 1% BSA, 0.1% NaN₃) and resuspended at 10⁶ cells/ml in PBS wash supplemented with 0.4% paraformaldehyde. Samples were analyzed on a FACStar^{PLUS} flow cytometer (Becton Dickinson, Mountain View, Calif.).

### T. gondii Infection

Mice were injected, i.p., with 0.5 mL PBS containing 20 cysts of the avirulent ME49 strain of Toxoplasma gondii, that had been prepared from the brains of infected C57 BL/6 mice. The mice were monitored daily.

For ex vivo cytokine analysis, single-cell spleen cell and peritoneal exudate cell cultures were isolated from infected mice, and contaminating red cells were removed using ACK lysing buffer (Bio Whittaker, Walkersville, Md.). The spleen cells and PECs were then cultured in 96 well plates, at 8×10⁵ and 4×10⁵ cells per well respectively, in 200 μl RPMI medium (Life Technologies, Gaithersberg, Md.) supplemented with 10% (v/v) fetal bovine serum (Life Technologies). In some cases, the cell cultures were stimulated with 10 mg/mL plate-bound anti-CD3 (Pharmingen, San Diego, Calif.) or 10 mg/mL STAg (soluble tachyzoite antigen), which had been prepared from sonicated RH parasites as previously described (Grunvald et al., Infect. Immun., 64:2010-2018, 1996). Conditioned media were collected 72 hours later for determination of IFNγ and IL-12 p40 levels using sandwich ELISA as described previously (Scharton-Kersten et al., J. Immunol., 157:4045-4054, 1996).

Sera were prepared from blood that was collected at the time of sacrifice, allowed to clot, and then centrifuged at 6000 rpm for 10 minutes.

L. monocytogeizes Infection

The mice were inoculated with different doses i.p. of the L. monocytogenes EGD strain (kindly provided by Dr. K Elkins, U.S. Food and Drug Administration). Health and survival of the mice were monitored daily for at least 14 days. For experiments involving the measurement of serum cytokine levels, bacterial loads in the spleen and liver, or IGTP expression levels, the mice were inoculated with 1000 bacteria i.p., and the sera and relevant tissues were isolated three days later. Bacterial counts were determined by excising spleens and livers sterilely, homogenizing portions of the organs in PBS, and plating serial dilutions of the homogenate on LB agar plates. Colony counts were determined the following day, and the total bacterial load per organ was calculated.

### MCMV Infection

MCMV stocks were generated by homogenizing the salivary glands of C57B1/6-129SvImJ mice that had been inoculated with the Smith MCMV strain (American Type Culture Collection VR-194) in 10% (v/v) fetal bovine serum (FBS, Hyclone, Logan, Uath)/Dulbecco's Modified Eagle's Medium (DMEM, Life Technologies, Gaithersberg, Md.) at 11 days after i.p. inoculation. The viral stocks were titered by infecting confluent lawns of primary embryonic fibroblasts in 6-well tissue culture plates, with dilutions of the viral lysates. The infected cells were overlaid with 1% agar (w/v) in 2% FBS/DMEM and then incubated 4-7 days. Plaque forming units (PFU's) were identified microscopically.

To assess host restriction of MCMV infection, mice were inoculated i.p. with 5×10⁴ PFU MCMV in 0.5 mL 10% FBS/DMEM. Three days later, spleen and liver samples were isolated and homogenized in the same medium, and used for PFU determination.

### Results

To target the LRG-47 and IRG-47 genes, we first isolated clones of the murine form of each gene, and used them to create targeting vectors in which the majority or all of the protein coding regions were deleted (FIGS. 6a and 6). Using standard techniques, the targeting vectors were then used to create mice that completely lacked expression of LRG-47 or IRG-47 (FIGS. 6c and 6). These LRG-47 and IRG-47-deficient mice displayed no obvious abnormalities; they were produced in normal numbers; and necropsies revealed no major alterations in tissue architecture. In addition, FACS analysis of splenocytes from adult mice revealed no changes in the development of T cell, B cell, macrophage, and natural killer cell populations (FIG. 7).

To assess the general importance of the two genes in host defense, the LRG-47 and IRG-47-deficient mice were challenged with three representative pathogens: Toxoplasma gondii, Listeria monocytogenes, and murine cytomegalovirus (MCMV). IFNγ is an absolute requirement for normal restriction of each pathogen, as illustrated by the fact that mice that lack production of IFNγ or its receptor demonstrate markedly increased susceptibility to each agent (Scharton-Kersten et al., J. Immunol., 157:4045-4054, 1996; Dalton et al., Science, 259:1739-1742, 1993; Huang et al., Science, 259:1742-1745, 1993; Presti et al., J. Exp. Med., 188:577-588, 1998). However, the IFNγ-induced pathways that mediate resistance to each pathogen have not been completely delineated. Expression of the IGTP family of proteins, including LRG-47 and IRG-47, is markedly increased following infection with each of the pathogens (FIG. 8); therefore, the genes are candidate mediators of immune defense against these agents.

In the first group of studies, the mice were challenged with T. gondii, an intracellular protozoan parasite (Yap & Sher, Immunobiology, 201:240-247, 1999). Its infection is characterized by an acute phase in which the rapidly proliferating organism, or tachyzoite, disseminates throughout the host, followed by a chronic phase in which the dormant organism, or bradyzoite, inhabits mainly central nervous tissue and muscle (Yap & Sher, Immunobiology, 201:240-247, 1999). While control of both phases requires production of IFNγ (Scharton-Kersten et al., J. Immunol., 157:4045-4054, 1996), the immune mechanisms that are active during each phase differ somewhat. For instance, tumor necrosis factor a and nitric oxide play important roles during the chronic phase, but not the acute phase (Yap & Sher, J. Ex. Med., 189:1083-1091, 1999). Following penetration into the host cell, T. gondii resides in a parasitophorous vacuole that resists interaction with the endocytic machinery of the cell, and consequently provides the organism with a safe environment in which to replicate (Sibley, Seinin Cell Biol., 4:335-344, 1993).

LRG-47-deficient, IRG-47-deficient, and wild-type mice were inoculated i.p. with 20 cysts of T. gondii, and their survival was followed for 40 days. Similar to what has been shown previously for IGTP-deficient mice (Taylor et al., Proc. Natl. Acad. USA, 97:751-755, 2000), LRG-47 deficient mice displayed a complete loss of resistance to the parasite during the acute phase: all LRG-47-deficient mice died between days 9 and 11 post-infection (FIG. 9a), the same time frame in which IFNγ-deficient mice succumb to the infection (Scharton-Kersten et al., J. Immunol., 157:4045-4054, 1996). The loss of defense in the LRG-47-deficient mice was not a result of decreased IFNγ production, as serum levels of the cytokine at 5 days post-infection were similar in LRG-47-deficient and wild-type mice, at 2611+/−2318 ρg/mL and 2143+/−1048, respectively. In addition, decreased resistance to T. gondii in the LRG-47-deficient mice was not simply a result of decreased IGTP production, as IGTP levels in splenocytes showed a robust increase after T. gondii infection in vivo. Similarly, IGTP expression is normal in LRG-47-deficient fibroblasts following stimulation with IFNγ in vitro (FIG. 6c). Therefore, IGTP and LRG-47 are independent factors that are both critically important for normal clearance of acute T. gondii infections.

In contrast to the IGTP-deficient and LRG-47-deficient mice, IRG-47-deficient mice displayed only marginally reduced resistance to T. gondii (FIG. 9b). Following challenge with 20 cysts of the parasite, only a portion of the IRG-47-deficient mice died, with death occurring mainly during the chronic phase, between days 10 and 47 post-infection (FIG. 5b). Furthermore, the burden of T. gondii cysts in the brains of IRG-47-deficient and wild-type mice at 33 days post-infection were found to be statistically the same. As expected, IFNγ levels increased normally in the IRG-deficient mice following an appropriate stimulation: peritoneal exudate cells from IRG-47-deficient mice produced 5164+/−1153 ρg/mL IFNγ after exposure to the soluble T. gonidii antigen STAg in vitro, as compared to 4558+/−3011 ρg/mL by wild-type cells. Thus as opposed to LRG-47 and IGTP, IRG-47 plays only a modest role in restricting acute T. gondii infections that does not become apparent until the chronic phase of infection.

In a second series of experiments, the mice were challenged with L. monocytogenes, a gram-positive bacterium that produces an acute infection (Schuchat & Broome, “Infections caused by Listeria monocytogenes.” In Robbins pathologic basis of disease. (eds. Cotran et al.) Saunders, Philadelphia, 899-901, 1994). As opposed to T. gondii, L. monocytogenes resides in a vacuole only briefly after entry into the host cell; rather it quickly lyses the vacuole to release itself into the cytosol where it replicates (Cossart & Lecuit, EMBO, 17:3797-3806, 1998). Cytosolic bacteria then trigger their spread into adjacent cells by contacting the host cell plasma membrane and forming protrusions from infected cells that are internalized by neighboring cells (Cossart & Lecuit, EMBO, 17:3797-3806, 1998). In this manner, the bacteria are able to remain intracellular as they disseminate; consequently cell-based imnune mechanisms, particularly those regulated by IFNγ are the predominant means of clearance by the host (Huang et al., Science, 259:1742-1745, 1993).

Previously it was shown that IGTP-deficient mice exhibit normal resistance to L. monocytogenes, as opposed to IFNγ-deficient mice that display greatly impaired resistance; therefore it was concluded that IGTP is not an essential factor in restricting growth of this bacterium (Taylor et al., Proc. Natl. Acad. USA, 97:751-755, 2000). For the current studies, LRG-47-deficient and IRG-47-deficient mice were infected with 1000 L. monocytogenes and then monitored for their survival (FIG. 10). The responses of the mice to this challenge were markedly different, with the LRG-47-deficient mice succumbing rapidly and displaying uniform death by 5 days, paralleling that seen in IFNγ-deficient mice (FIG. 10a), while the IRG-47-deficient showed no adverse effects and survived for 40 day observation period (FIG. 10b). The ability of the LRG-47 KO mice to defend against L. monocytogenes was examined further by measuring the bacterial burdens in spleen and liver at three days following inoculation (FIG. 10c). In both tissues, there were substantially more bacteria present in LRG-47-deficient mice than in wild-type mice. Thus in summary, LRG-47 is an essential factor in mediating IFNγ-induced clearance of L monocytogenes, whereas IRG-47 and IGTP are dispensable.

In a final series of studies, the response of the mice to MCMV was characterized. MCMV is a double-stranded DNA herpes virus that, in an immunocompetent host, establishes a chronic infection characterized by latency and intermittent viral shedding (Hirsch, “Cytomegalovirus infection.” In Robbins pathologic basis of disease. (eds. Cotran etal.) Saunders, Philadelphia, 794-797, 1994). However in an immunocompromised setting, the virus can produce an acute infection with significant mortality. IFNγ that is produced soon after infection by NK cells and subsequently by CD4 T cells, is an important element of viral clearance, as it activates macrophages, enhances cytotoxic CD8 T cells activity, and inhibits viral replication and gene expression (Heise & Virgin, J. Virol., 69:904-909, 1995). The significance of the IFNγ response is demonstrated by neutralizing IFNγ prior to MCMV infection, leading to increased viral loads in tissues and increased mortality (Orange et al., J. Exp. Med., 182:1045-1056, 1995).

LRG-47-deficient and IRG-47-deficient mice were inoculated with MCMV, and at 3 days after inoculation, the loads of MCMV in tissues of the mice were determined. In both spleen (not shown) and liver (FIG. 11), comparable numbers of viral plaque-forming units were detected in wild-type mice, and in LRG-47 (not shown) and IRG-47-deficient mice. Similar results were reported previously for IGTP-deficient mice (Taylor et al., Proc. Natl. Acad. USA, 97:751-755, 2000). Therefore, LRG-47, IRG-47, and IGTP may not be critical factors for defense against MCMV.

### Discussion

IFNγ mediates a broad range of antimicrobial responses that are necessary for host clearance of protozoa, bacteria, and certain viruses. As summarized in Table 1, the studies presented here for LRG-47 and IRG-47, and those presented previously for IGTP, demonstrate that members of the IGTP protein family are crucial elements of an IFNγ-regulated antimicrobial program. Furthermore, they suggest that each gene in the family provides a distinct, non-redundant element of immune defense.

TABLE 1

| | T. gondii | L. monocytogenes | MCMV |
|---|---|---|---|
| IFNγ KO | S (acute/chronic) | S | S |
| IGTP KO | S (acute) | normal | normal |
| LRG-47 KO | S (acute) | S | normal |
| IRG-47 KO | S (chronic) | normal | normal |

KO = knockout
S = susceptible

Host defense in the LRG-47, IRG-47, and IGTP-deficient mice has only been examined against a limited number of pathogens. However, it is clear that the proteins are critical for defense against some intracellular pathogens that form vacuoles, including T. gondii that persists in a vacuole after infecting the cell, and L. monocytogenes that transiently inhabits a vacuole after infecting the cell. Pathogen containing vacuoles (PCVs) are isolated from the normal lipid trafficking in the cell, in that they do not fuse with lysosomes and become acidified, and consequently in this context, the pathogen can safely inhabit the cell. However, if trafficking to PCVs is altered to effect vacuole maturation, then survival of the pathogen is compromised. LRG-47, IRG-47, and IGTP may initiate intracellular pathogen killing by regulating trafficking to the PCV. These proteins are GTP-binding protein that localize to the ER of cells, and consequently it was proposed previously that they regulate lipid/protein trafficking within the cell. Taking this and the fact that they profoundly influence the survival of some vacuolar pathogens, it is possible that they do, in fact, regulate the maturation of PCVs at some level. Parallels can be drawn between the IGTP protein family and the rab family of GTP-binding proteins, which also regulate vesicular trafficking in the cell. Rab5 associates with the early endosomal compartment and rab7 with the late endosomal compartment, and both are thought to modulate trafficking to PCVs and effect the killing of pathogens including L. monocytogenes and S. typhimurium. The studies presented here suggest that another family of GTP-binding proteins in a compartment more distal to the vacuole, the ER, may also regulate vacuole maturation.

Individually, LRG-47, IRG-47, and IGTP apparently do not play an important role in restricting infections of the herpes virus MCMV. It remains possible that the proteins, however, that they could act in concert to regulate clearance of MCMV, or that they could regulate clearance of other viruses. Previously it has been suggested that another protein within the IGTP protein family, TGTP, may regulate clearance of vesicular stomatitis virus (VSV). In those studies, overexpression of TGTP in fibroblasts blocked cellular lysis by VSV; however, interpretation was complicated by the fact that overexpression of TGTP was toxic to the cells. Yet it remains possible that members of the IGTP family could also be involved in viral clearance.

### III. EXAMPLES

### Example 1

Nucleotide and Amino Acid Sequence Variants of IGTP-fanily Proteins

Variant IGTP-family proteins include proteins that differ in amino acid sequence from the prototypical sequences (IGTP: GenBank Accession No. U53219; IRG-47: Accession M63630; LRG-47: Accession U19119) but that share at least 70% amino acid sequence homology with this protein sequence, and that maintain immune-modulating activity. Other variants will share at least 75%, at least 80%, at least 90%, at least 95%, or at least 98% amino acid sequence homology. Manipulation of the nucleotide sequence of IGTP using standard procedures, including for instance site-directed mutagenesis or PCR, can be used to produce such variants. The simplest modifications involve the substitution of one or more amino acids for amino acids having similar biochemical properties. These so-called conservative substitutions are likely to have minimal impact on the activity of the resultant protein. Table 2 shows amino acids that may be substituted for an original amino acid in a protein, and which are regarded as conservative substitutions.

TABLE 2

| Original Residue | Conservative Substitutions |
|---|---|
| Ala | ser |
| Arg | lys |
| Asn | gln; his |
| Asp | glu |
| Cys | ser |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

More substantial changes in enzymatic function or other protein features may be obtained by selecting amino acid substitutions that are less conservative than those listed in Table 2. Such changes include changing residues that differ more significantly in their effect on maintaining polypeptide backbone structure (e.g., sheet or helical conformation) near the substitution, charge or hydrophobicity of the molecule at the target site, or bulk of a specific side chain. The following substitutions are generally expected to produce the greatest changes in protein properties: (a) a hydrophilic residue (e.g., seryl or threonyl) is substituted for (or by) a hydrophobic residue (e.g., leucyl, isoleucyl, phenylalanyl valyl or alanyl); (b) a cysteine or proline is substituted for (or by) any other residue; (c) a residue having an electropositive side chain (e.g., lysyl, arginyl, or histadyl) is substituted for (or by) an electronegative residue (e.g., glutamyl or aspartyl); or (d) a residue having a bulky side chain (e.g., phenylalanine) is substituted for (or by) one lacling a side chain (e.g., glycine).

Particular regions of IGTP-family proteins are known to be conserved, when the protein is compared to other members of the 47-48 kDa, IFNγ-induced GTPases (see, Taylor et al., J. Biol. Chem., 271:20399-20405, 1996 and Boehm et al., J. Immunol., 161:6715-6723, 1998, herein incorporated by reference in their entirety). These residues may be involved in and important to the functionality of the protein. As such, when variants are generated, and it is desired to maintain the function of the protein, it may be advantageous to avoid altering highly conserved residues. Such highly conserved residues include but are not necessarily limited to the active site(s) of the IGTP-family protein (including those regions of the protein commonly referred to as “consensus GTP-binding motifs”) that is responsible for GTPase activity.

Variant IGTP-family protein encoding sequences may be produced by standard DNA mutagenesis techniques, for example, M13 primer mutagenesis. Details of these techniques are provided in Sambrook et al. (In Molecular Cloning: A Laboratory Manual, CSHL, New York, 1989), Ch. 15. By the use of such techniques, variants may be created which differ in minor ways from the sequence of a prototypical IGTP-family member. The use of DNA molecules and nucleotide sequences that are derivatives of the prototypical sequences (e.g., GenBank Accession Numbers U53219, M63630, and U19119), and which differ from those by the deletion, addition, or substitution of nucleotides while still encoding a protein that has at least 70% sequence identity with a prototypical IGTP-family member and maintains immune-modifying activity, are comprehended by this invention. Also comprehended are more closely related nucleic acid molecules that share at least 75%, at least 80%, at least 90%, at least 95%, or at least 98% nucleotide sequence homology with a prototypical IGTP family member. In their most simple form, such variants may differ from this sequence by alteration of the coding region to fit the codon usage bias of the particular organism into which the molecule is to be introduced Such variants may contain one mutation, or two or more mutations, up to several mutations, so long as an immune-modifying activity of the protein is maintained.

Alternatively, the coding region may be altered by taking advantage of the degeneracy of the genetic code to alter the coding sequence such that, while the nucleotide sequence is substantially altered, it nevertheless encodes a protein having an amino acid sequence substantially similar to the IGTP protein sequences. For example, the 22nd amino acid residue of the murine IGTP protein (GenBank Accession Number U53219) is alanine. The nucleotide codon triplet GCA encodes this alanine residue. Because of the degeneracy of the genetic code, three other nucleotide codon triplets—GCG, GCC and GCT—also code for alanine. Thus, the nucleotide sequence of the murine IGTP could be changed at this position to any of these three alternative codons without affecting the amino acid composition or characteristics of the encoded protein. Based upon the degeneracy of the genetic code, variant DNA molecules may be derived from the cDNA and gene sequences disclosed herein using standard DNA mutagenesis techniques as described above, or by synthesis of DNA sequences. Thus, this invention also encompasses the use of nucleic acid sequences that encode an IGTP protein, but which vary from the disclosed nucleic acid sequences by virtue of the degeneracy of the genetic code, and proteins produced from such nucleic acid molecules.

Variants of an IGTP-family protein may also be defined in terms of their sequence identity with a prototype IGTP-family protein (such as murine IGTP, GenBank Accession Number U53219). As described above, IGTP proteins share at least 70%, at least 75%, at least 80%, at least 90%, at least 95%, or at least 98% amino acid sequence identity with this IGTP protein. Nucleic acid sequences that encode such proteins may readily be determined simply by applying the genetic code to the amino acid sequence of a putative IGTP protein, and such nucleic acid molecules may readily be produced by assembling oligonucleotides corresponding to portions of the sequence.

Nucleic acid molecules that are derived from the IGTP cDNA nucleic acid sequence include molecules that hybridize under stringent conditions to the prototypical IGTP nucleic acid molecules, or fragments thereof. Stringent conditions are hybridization at 65° C. in 6×SSC, 5×Denhardt's solution, 0.5% SDS and 100 μg sheared salmon testes DNA, followed by 15-30 minute sequential washes at 65° C. in 2×SSC, 0.5% SDS, followed by 1×SSC, 0.5% SDS and finally 0.2×SSC, 0.5% SDS.

Low stringency hybridization conditions (to detect less closely related homologs) are performed as described above but at 50° C. (both hybridization and wash conditions); however, depending on the strength of the detected signal, the wash steps may be terminated after the first 2×SSC wash.

IGTP nucleic acid encoding molecules, and orthologs and homologs of these sequences may be incorporated into transformation or expression vectors.

Likewise, variants or fragments of other IGTP-family proteins may be useful in the methods described herein. Such variants or fragments can be prepared essentially using methods that have been described for IGTP fragments and variants.

### Example 2

### IGTP Encoding Sequences in Other Animal Species

Considering the importance of IGTP to the murine response to infection, particularly intracellular infectious agents such as infection by parasites and bacteria, homologs of IGTP-family genes are likely to be present in a number of animal species, especially those susceptible to similar infections. With the provision herein of a native function of several prototypical murine IGTP-family proteins (including IGTP, LRG-47, and IRG-47), the benefit of cloning cDNAs and genes that encode IGTP-family protein homologs in other animal species is now apparent. Standard methods can be used.

As described above, homologs of the disclosed murine IGTP-family proteins have IGTP-family protein immune-modifying activity and typically possess at least 60% sequence identity counted over the full length alignment with the amino acid sequence of prototypical murine genes as described herein. Proteins with even greater similarity to the murine sequence will show greater percentage identities when assessed by this method, such as at least 70%, at least 75%, at least 80%, at least 90% or at least 95% sequence identity.

Both conventional hybridization and PCR amplification procedures may be utilized to clone sequences encoding IGTP-family protein homologs. Common to these techniques is the hybridization of probes or primers derived from the murine IGTP cDNA or gene sequence to a target nucleotide preparation. This target may be, in the case of conventional hybridization approaches, a cDNA or genomic library or, in the case of PCR amplification, a cDNA or genomic library, or an mRNA preparation. In particular, it may be advantageous to screen one or more tissue-specific cDNA libraries, for instance a human thymus or spleen library.

Direct PCR amplification may be performed on genomic or cDNA (e.g., thymus or spleen) libraries prepared from the animal species in question, or RT-PCR may be performed using mRNA extracted from the animal cells using standard methods. PCR primers will comprise at least 15 consecutive nucleotides of the murine IGTP cDNA or gene. One of ordinary skill in the art will appreciate that sequence differences between the murine IGTP cDNA or gene and the target nucleic acid to be amplified may result in lower amplification efficiencies. To compensate for this difference, longer PCR primers or lower annealing temperatures may be used during the amplification cycle. Where lower annealing temperatures are used, sequential rounds of amplification using nested primer pairs may be necessary to enhance amplification specificity. In addition, alignment and comparison of the encoding sequences of multiple IGTP-family proteins (see, e.g., Taylor et al., J. Biol. Chem., 271:20399-20405, 1996; Boehm et al., J. Immunol., 161:6715-6723, 1998) will provide conserved regions of the protein, thereby enabling the selection of regions from which it will be more advantageous generate primers or probes.

For conventional hybridization techniques, the hybridization probe is preferably conjugated with a detectable label such as a radioactive label, and the probe is preferably of at least 20 nucleotides in length. As is well known in the art, increasing the length of hybridization probes tends to give enhanced specificity. The labeled probe derived from the murine cDNA or gene sequence may be hybridized to an animal cDNA (for instance, human thymus or spleen) or genomic library and the hybridization signal detected using means known in the art. The hybridizing colony or plaque (depending on the type of library used) is then purified and the cloned sequence contained in that colony or plaque isolated and characterized.

Homologs of the murine IGTP-family proteins may alternatively be obtained by immunoscreening an expression library. Antibodies specific for the murine IGTP may be generated through conventional means; methods of raising antibodies are well known in the art (see Example 5, below). Such antibodies can be used to screen an expression cDNA library produced from the animal from which it is desired to clone the IGTP homolog, using routine methods. The selected cDNAs can be confirmed by sequencing.

### Example 3

### Expression of IGTP-family Proteins

With the provision herein of the immunological functions of IGTP-family proteins, advantages of the expression and purification of the IGTP-family proteins by standard laboratory techniques are now apparent and enabled. After expression, the purified IGTP-family protein or polypeptide may be used for functional analyses, antibody production, diagnostics, and patient therapy. Furthermore, the DNA sequence of the IGTP-family cDNA and its antisense strand can be manipulated in studies to understand the expression of the gene and to further elucidate the function of its product. Mutant forms of IGTP-family members may be isolated based upon information contained herein, and may be studied in order to detect alteration in expression patterns in terms of relative quantities, tissue specificity and functional properties of the encoded mutant IGTP-family protein. Partial or full-length cDNA sequences, which encode for the subject protein, may be ligated into bacterial expression vectors. Methods for expressing large amounts of protein from a cloned gene introduced into Escherichia coli (E. coli) may be utilized for the purification, localization and functional analysis of proteins. For example, fusion proteins consisting of amino terminal peptides encoded by a portion of the E. coil lacZ or trpE gene linked to IGTP-family proteins may be used to prepare polyclonal and monoclonal antibodies against these proteins. Thereafter, these antibodies may be used to purify proteins by immunoaffinity chromatography, in diagnostic assays to quantitate the levels of protein and to localize proteins in tissues and individual cells by immnunofluorescence.

Intact native protein may also be produced in E. coli in large amounts, e.g., for functional studies. Methods and plasmid vectors for producing fusion proteins and intact native proteins in bacteria are described in Sambrook et al. (Sambrook et al., In Molecular Cloning: A Laboratory Manual, Ch. 17, CSHL, New York, 1989). Such fusion proteins may be made in large amounts, are easy to purify, and can be used to elicit antibody response. Native proteins can be produced in bacteria by placing a strong, regulated promoter and an efficient ribosome-binding site upstream of the cloned gene. If low levels of protein are produced, additional steps may be taken to increase protein production; if high levels of protein are produced, purification is relatively easy. Methods are presented in Sambrook et al. (Molecular Cloning: A Laboratory Manual, CSHL, New York, 1989) and are well known in the art. Often, proteins expressed at high levels are found in insoluble inclusion bodies. Methods for extracting proteins from these aggregates are described by Sambrook et al. (Molecular Cloning: A Laboratory Manual, Ch. 17, CSHL, New York, 1989). Vector systems suitable for the expression of lacZ fusion genes include the pUR series of vectors (Ruther and Muller-Hill, EMBO J. 2:1791, 1983), pEX1-3 (Stanley and Luzio, EMBO J. 3:1429, 1984) and pMR100 (Gray et al., Proc. Natl. Acad. Sci. USA 79:6598, 1982). Vectors suitable for the production of intact native proteins include pKC30 (Shimatake and Rosenberg, Nature 292:128, 1981), pKK177-3 (Amann and Brosius, Gene 40:183, 1985) and pET-3 (Studiar and Moffatt, J. Mol. Biol. 189:113, 1986). IGTP proteins may be isolated from protein gels, lyophilized, ground into a powder and used as an antigen. The DNA sequence can also be transferred from its existing context to other cloning vehicles, such as other plasmids, bacteriophages, cosmids, animal viruses and yeast artificial chromosomes (YACs) (Burke et al., Science 236:806-812, 1987). These vectors may then be introduced into a variety of hosts including somatic cells, and simple or complex organisms, such as bacteria, fungi (Timberlake and Marshall, Science 244:1313-1317, 1989), invertebrates, plants (Gasser and Fraley, Science 244:1293, 1989), and animals (Pursel et al., Science 244:1281-1288, 1989), which cell or organisms are rendered transgenic by the introduction of the heterologous IGTP cDNA.

For expression in mammalian cells, the cDNA sequence may be ligated to heterologous promoters, such as the simian virus (SV) 40 promoter in the pSV2 vector (Mulligan and Berg, Proc. Natl. Acad. Sci. USA 78:2072-2076, 1981), and introduced into cells, such as monkey COS-1 cells (Gluzman, Cell 23:175-182, 1981), to achieve transient or long-term expression. The stable integration of the chimeric gene construct may be maintained in mammalian cells by biochemical selection, such as neomycin (Southern and Berg, J. Mol. Appl. Genet. 1:327-341, 1982) and mycophenolic acid (Mulligan and Berg, Proc. Natl. Acad. Sci. USA 78:2072-2076, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR.

The cDNA sequence (or portions derived from it) or a mini gene (a cDNA with an intron and its own promoter) may be introduced into eukaryotic expression vectors by conventional techniques. These vectors are designed to permit the transcription of the cDNA in eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan et al., Proc. Natl. Acad. Sci. USA 78:1078-2076, 1981; Gorman et al., Proc. Natl. Acad. Sci USA 78:6777-6781, 1982). The level of expression of the cDNA can be manipulated with this type of vector, either by using promoters that have different activities (for example, the baculovirus pAC373 can express cDNAs at high levels in S. frugiperda cells (Summers and Smith, In Genetically Altered Viruses and the Environment, Fields et al. (Eds.) 22:319-328, CSHL Press, Cold Spring Harbor, N.Y., 1985) or by using vectors that contain promoters amenable to modulation, for example, the glucocorticoid-responsive promoter from the mouse mammary tumor virus (Lee et al., Nature 294:228, 1982). The expression of the cDNA can be monitored in the recipient cells 24 to 72 hours after introduction (transient expression).

In addition, some vectors contain selectable markers such as the gpt (Mulligan and Berg, Proc. Natl. Acad. Sci. USA 78:2072-2076, 1981) or neo (Southern and Berg, J. Mol. Appl. Genet. 1:327-341, 1982) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Sarver et al., Mol. Cell Biol. 1:486, 1981) or Epstein-Barr (Sugden et al., Mol. Cell Biol. 5:410, 1985). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., J. Biol. Chem. 253:1357, 1978).

The transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb, Virology 52:466, 1973) or strontium phosphate (Brash et al., Mol. Cell Biol. 7:2013, 1987), electroporation (Neumann et al., EMBO J 1:841, 1982), lipofection (Feigner et al., Proc. Natl. Acad. Sci USA 84:7413, 1987), DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-2167, 1980), or pellet guns (Klein et al., Nature 327:70, 1987). Alternatively, the cDNA, or fragments thereof, can be introduced by infection with virus vectors. Systems are developed that use, for example, retroviruses (Bernstein et al., Gen. Engr'g 7:235, 1985), adenoviruses (Ahmad et al., J. Virol. 57:267, 1986), or Herpes virus (Spaete et al., Cell 30:295, 1982). IGTP-family protein encoding sequences can also be delivered to target cells in vitro via non-infectious systems, for instance liposomes.

These eukaryotic expression systems can be used for studies of IGTP-family protein encoding nucleic acids and mutant forms of these molecules, the IGTP-family protein and mutant forms of this protein. Such uses include, for example, the identification of regulatory elements located in the 5′ region of IGTP-family genes on genomic clones that can be isolated from genomic DNA libraries. The eukaryotic expression systems may also be used to study the function of the normal complete protein, specific portions of the protein, or of naturally occurring or artificially produced mutant proteins.

Using the above techniques, the expression vectors containing an IGTP-family gene sequence or cDNA, or fragments or variants or mutants thereof, can be introduced into human cells, mammalian cells from other species or non-mammalian cells as desired. The choice of cell is determined by the purpose of the treatment. For example, monkey COS cells (Gluzman, Cell 23:175-182, 1981) that produce high levels of the SV40 T antigen and permit the replication of vectors containing the SV40 origin of replication may be used. Similarly, Chinese hamster ovary (CHO), mouse NIH 3T3 fibroblasts or human fibroblasts or lymphoblasts may be used.

The present invention thus encompasses recombinant vectors that comprise all or part of an IGTP-family gene or cDNA sequences for expression in a suitable host in order to enhance or down-regulate an immune response of that cell, more particularly an immune response to an infectious agent. The IGTP-family DNA is operatively linked in the vector to an expression control sequence in the recombinant DNA molecule so that the corresponding IGTP-family polypeptide can be expressed. The expression control sequence may be selected from the group consisting of sequences that control the expression of genes of prokaryotic or eukaryotic cells and their viruses and combinations thereof. The expression control sequence may be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

The host cell, which may be transfected with the vector of this invention, may be selected from the group consisting of E. coli, Pseudomonas, Bacillus subtilis, Bacillus stearothennophilus or other bacilli; other bacteria; yeast; fingi; insect; mouse or other animal; or plant hosts; or human tissue cells.

It is appreciated that for mutant or variant IGTP-family member-encoding DNA sequences, similar systems are employed to express and produce the mutant product. In addition, fragments of IGTP-famnily proteins can be expressed essentially as detailed above. Such fragments include individual IGTP-family protein domains or sub-domains, as well as shorter fragments such as peptides. IGTP-family protein fragments having therapeutic properties (e.g., anti-infectious properties or immune modifying activities) may be expressed in this manner also.

Measurements of the amount of specific proteins (e.g. IGTP, LRG-47, IRG-47, and so forth) may be carried out through many techniques well known to those of ordinary skilled in the art. These include quantitative immunoblot analysis, as well as enzyme activity assays.

Quantitative immunoblot analysis refers to a method of measuring the actual amount of a stable protein present in a cell or cell fraction or other sample. Such analysis is well known in the art, and is described for instance in Scott and Klionsky (J. Cell Biol. 131:1727-1735, 1995). In general, proteins from cells expressing the protein of interest are precipitated using trichloracetic acid, then resuspended in SDS-sample buffer and subjected to polyacrylamide gel electrophoresis (PAGE) to separate individual proteins by size. The resultant gel is then electrophoretically transferred (“western blotted”) to a nitrocellulose sheet or other equivalent substrate, and subjected to immunoblot analysis using antibodies (either monoclonal or polyclonal) to the protein(s) of interest. See Sambrook et al. (In Molecular Cloning: A Laboratoiy Manual, Cold Spring Harbor, N.Y., 1989) for general techniques of western blotting. It is advantageous to use polyclonal or monoclonal antibody to the engineered IGTP protein to probe the western blot. Optionally, though especially if the engineered and endogenous proteins are of similar size, an epitope tag can be added to the engineered protein for differential detection. The use of epitope tags is well known.

Primary antibody binding is detected using a secondary antibody, which itself is chemically linked to an indicator molecule. The indicator molecule can be an enzymatically active protein that catalyzes a reaction, the end product of which produces fluorescence. The relative amount of each protein (e.g., IGTP, LRG-47, IRG-47, and so forth) in different cell fractions is then calculated based on densitometric measurement of the fluorescence signal recorded on exposed x-ray film. Protein standards of known subcellular localization may be used for comparison.

One of ordinary skill in the art will recognize that many other techniques could be used to measure the amount of a protein present in a sample or a cell. For instance, the amount of IGTP protein (or another IGTP-family protein) in a cell or other sample could be measured using a quantitative enzyme-linked immunosorbant assay (‘ELISA’) as described by Aboagye-Mathiesen et al. (Placenta 18:155-61, 1997). Presentation of the above example is not meant to limit the invention to the method discussed.

### Example 4

### Suppression of Protein Expression and/or Activity

A reduction of IGTP-family protein expression in a transgenic cell may be obtained by introducing into cells an antisense construct based on the IGTP-family member encoding sequence (e.g., GenBank Accession Number U53219, M63630, and U19119) or gene sequence or associated regulatory nucleotide sequences. For antisense suppression, a nucleotide sequence from the IGTP encoding region, e.g. all or a portion of the IGTP cDNA or gene, is arranged in reverse orientation relative to the promoter sequence in the transformation vector. Other aspects of the vector may be chosen as discussed above (Example 3).

The introduced antisense sequence need not be the full length IGTP-family member cDNA or gene, and need not be exactly homologous to the equivalent sequence found in the cell type to be transformed. Generally, however, where the introduced sequence is of shorter length, a higher degree of homology to the native IGTP-family sequence will be needed for effective antisense suppression. The introduced antisense sequence in the vector may be at least 30 nucleotides in length, and improved antisense suppression will typically be observed as the length of the antisense sequence increases. The length of the antisense sequence in the vector advantageously may be greater than 100 nucleotides. For suppression of the IGTP gene, for instance, transcription of an antisense construct results in the production of RNA molecules that are the reverse complement of mRNA molecules transcribed from the endogenous IGTP gene in the cell.

Although the exact mechanism by which antisense RNA molecules interfere with gene expression has not been elucidated, it is believed that antisense RNA molecules bind to the endogenous mRNA molecules and thereby inhibit translation of the endogenous mRNA.

Suppression of endogenous IGTP-family member expression can also be achieved using ribozymes. Ribozymes are synthetic RNA molecules that possess highly specific endonbonuclease activity. The production and use of ribozymes are disclosed in U.S. Pat. No. 4,987,071 to Cech and U.S. Pat. No. 5,543,508 to Haselhoff. The inclusion of ribozyme sequences within antisense RNAs may be used to confer RNA cleaving activity on the antisense RNA, such that endogenous mRNA molecules that bind to the antisense RNA are cleaved, which in turn leads to an enhanced antisense inhibition of endogenous gene expression.

Finally, dominant negative mutant forms of the disclosed sequences may be used to block endogenous IGTP activity.

Suppression of IGTP-family member expression can be, for instance, used to treat auto-immune disorders caused by abnormalities in the corresponding gene. In general, such auto-immune disorders are those that involve an over-stimulation or mis-regulation of the Th1 immune response system, such as irritable bowel disease (IBD), rheumatoid arthritis, auto-immune diabetes mellitus, lupus erythematosis, sarcoidosis, multiple sclerosis, chronic delayed type hypersensitivity (DTH), and auto-immune encephalomyelitis.

Alternatively, the activity of an IGTP-family protein can be suppressed by administering specific binding agents, such as monoclonal antibodies, that bind to and interfere with a biological activity of the IGTP-family protein. See Example 5 for a disclosure about how to make such specific binding agents.

The following description provides broad enablement for a variety of antisense and antigene technologies that can be used to target a protein encoding sequence, such as an IGTP-family member encoding sequence identified in this specification.

A. Regulation at the Nucleic Acid Level

The natural mechanism for producing proteins in living cells starts with the DNA being transcribed into RNA. The resulting RNA molecule is then translated into a protein. This chain of events (DNA→RNA→Protein) allows for the regulation of the protein at three different levels. At the first level of regulation the DNA can be targeted. This is done such that transcription is inhibited. For example, a small circular oligonucleotide molecule can be placed in contact with the DNA thus inhibiting and/or altering transcription (Wolf, Nature Biotechnology 16,341-344, 1998). At the next level the translation of the RNA can be inhibited. This can be done through the use of complementary oligonucleotides or oligonucleotide analogs that bind to the target RNA molecule. In some instances these polynucleotide or analog molecules can be designed so that they are catalytic. In other words, they can be designed so that they can bind to a first target RNA, cleave it, and then move on to cleave a second RNA.

One of ordinary skill in the art will appreciate that antisense and sense molecules can be designed, and produced in many different ways, some of which are discussed in the following sections of this specification.

B. The Design of Antisense and Sense Molecules and Catalytic Nucleic Acid Molecules

To inhibit the transcription, and translation of the target molecule, the antisense or sense molecule must persist in the cell for a sufficient period to contact the target nucleic acid. It is therefore often desirable to engineer the antisense or sense molecules to be nuclease resistant so that they persist for a longer period of time in the cell. This can be done, for example, by substituting the normally occurring phosphodiester linkage that connects the individual bases of the antisense or sense molecule with modified linkages. These modified linkages may, for example, be a phosphorothioate, methylphosphonate, phosphodithioate, or phosphoselenate. Furthermore, a single antisense molecule may contain multiple substitutions in various combinations.

The molecule can also be designed to contain different sugar molecules. For example the molecule may contain the sugars ribose, deoxyribose or mixtures thereof, which are linked to a base. The bases give rise to the molecules' ability to bind complementarily to the target RNA. Complementary binding occurs when the base of one molecule forms a hydrogen bond with another molecule. Normally the base adenine (A) is complementary to thymidine (T) and uracil (U), while cytosine (C) is complementary to guanine (G). Therefore, the sequence 5′-ACGA-3′ of the antisense molecule will bind to 5′-UCGU-3′ of the target RNA, or 5′-TCGT-3′ of the target DNA. Additionally, in order to be effective, the antisense and sense molecules do not have to be 100% complementary to the target RNA or DNA.

The antisense and sense polynucleotides can vary in length. Generally, a longer complementary region will give rise to a molecule with higher specificity. However, these longer molecules tend to be harder to produce synthetically. Therefore, the longer molecules are most often used in conjunction with systems that produce the therapeutic molecules in vivo. Such systems can involve cloning the sequence into a vector, and then delivering the vector to a host cell. The host cell then supplies the necessary components for transcription of the therapeutic molecule. Shorter polynucleotides (such as oligonucleotides and their analogs) can conveniently be produced synthetically as well as in vivo. The oligonucleotides can be DNA or RNA, or chimeric mixtures or derivatives or modified versions thereof, single-stranded or double-stranded. The oligonucleotide can be modified at the base moiety, sugar moiety, or phosphate backbone, and may include other appending groups such as peptides, or agents facilitating transport across the cell membrane (see, e.g., Letsinger et al., PNAS USA 86:6553-6556, 1989; Lemaitre et al., PNAS USA 84:648-652, 1987; PCT Publication No. WO 88/09810) or blood-brain barrier (see, e.g., PCT Publication No. WO 89/10134), hybridization triggered cleavage agents (see, e.g. Krol et al., BioTechiniques 6:958-976, 1988) or intercalating agents (see, e.g., Zon, Pharm. Res. 5:539-549, 1988).

In a particular aspect of the invention, the antisense or sense polynucleotide is a single-stranded DNA (ssDNA), although it is not limited to this. In a more particular aspect, such a polynucleotide includes a sequence antisense or sense to the nucleic acid sequence of GenBank Accession Number U53219, or the complementary sequence thereof. The oligonucleotide may be modified at any position on its structure with substitutes generally known in the art. For example, a modified base moiety may be 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil methoxyaminomethyl-2-thiouracil, beta-D-mannosylqueosine, 5′-methoxycarboxymethyluracil, 5-methoxyuracil, 2-methylthio-N˜6-isopentenyladenine, uracil-5-oxyacetic acid, pseudouracil queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil and 2,6-diaminopurine.

In another embodiment, the polynucleotide includes at least one modified sugar moiety such as arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Catalytic nucleic acid sequences can be designed which degrade target sequences. Such catalytic nucleic acid sequences can contain complementary regions that specifically hybridize to the target sequence, and non-complementary regions, which typically contain a sequence that gives the molecule its catalytic activity. Since catalytic molecules are subject to the same potential problem of degradation as other antisense or sense molecules, the catalytic molecules can be designed to contain the same substitutions already discussed.

A particular type of catalytic nucleic acid molecule is a ribozyme, which may be used to inhibit gene expression. Ribozymes may be synthesized and administered to the subject, or may be encoded on an expression vector, from which the ribozyme is synthesized in the targeted cell (as in PCT publication WO 9523225, and Beigelman et al. Nucl. Acids Res. 23:4434-4442, 1995). Examples of oligonucleotides with catalytic activity are described in WO 9506764, WO 9011364, and Sarver et al., Science 247:1222-1225, 1990.

Conjugates with antisense nucleic acid sequences can also be used to degrade RNA. For example, conjugates complexed with metal groups, e.g. terpyridylCu (II), are capable of mediating mRNA hydrolysis (Bashkin et al., Appl. Biochem Biotechnol. 54:43-56,1995).

The relative ability of an oligomer such as a polynucleotide to bind to a complementary strand is compared by determining the melting temperature of a hybridization complex of a polypeptide and its complementary strand. The melting temperature (Tₘ), a characteristic physical property of double helices, denotes the temperature in degrees Centigrade at which 50% helical versus coiled (unhybridized) forms are present. Base stacking, which occurs during hybridization, is accompanied by a reduction in UV absorption (hypochromicity). A reduction in UW absorption indicates a higher Tₘ. The higher the Tₘ the greater the strength of the binding of the hybridized strands. Generally, 100% complementarity between two nucleic acid sequences achieves optimal hybridization of a polynucleotide to its target RNA.

C. The Production of Antisense and Sense Molecules and Catalytic Nucleic Acid Molecules

Polynucleotides (and analogs) may be synthesized by standard methods known in the art, for example by use of an automated DNA synthesizer. Several different models of machines for synthesizing oligonucleotides are available (e.g. from Perkin-Elmer Applied Biosystems, 850 Lincoln Centre Drive, Foster City, Calif. 94404; Biosearch, Applied Biosystems, etc.). Furthermore, these machines can be programmed to allow the synthesis of oligonucleotides that contain the modified linkages mentioned above. After synthesis the oligonucleotides can be purified through capillary electrophoresis or other chromatography techniques. Phosphorothioate oligos may be synthesized by the method of Stein et al. (Nucl. Acids Res. 16:3209, 1998), methylphosphonate oligos can be prepared by use of controlled pore glass polymer supports (Sarin et al., PNAS USA 85:7448-7451, 1988).

Another method of producing the antisense, sense, and catalytic nucleic acid molecules is the use of PCR. This particular method has the capability of generating longer polynucleotides than those normally produced through synthesis. This method involves tagging either the sense or antisense primer with an appropriate moiety, such as biotin. The moiety will then be used in subsequent purification. This allows the molecule that is complementary to the target sequence to be extracted from the complementary strand.

Alternatively, a vector which contains the DNA sequence encoding the therapeutic nucleic acid molecule can be used. Once the vector is delivered to the host cell, the cell will transcribe the DNA into the antisense or catalytic nucleic acid molecule. If the host cell is that of the subject, this method accomplishes production and delivery of the molecule simultaneously.

D. Peptide Nucleic Acids (PNAs)

A peptide nucleic acid (PNA) (Nielsen et al., Science 254:1497-500, 1991; Wittung et al., Nature 368:561-3, 1994; Hanvey et al., Science 258:1481-5, 1992; and Egholm et al., Nature 365:566-8, 1993) is a DNA or RNA mimic. A PNA molecule is made up of monomers, which generally include ligands (such as a base) linked to a peptide backbone. PNA molecules can interfere with expression of a target sequence, and bind to dsDNA, ssDNA or RNA, for example, through Watson-Crick base pair formation, or can form a triple helix with double stranded molecules through Hoogsteen base pair formation. PNA molecules can also induce strand invasion, displacing the strands of dsDNA, and annealing to one of the strands. For instance, if the PNA molecule is a sense molecule, it can induce strand invasion, separating the strands of dsDNA, and anneal to the minus strand.

Monomers useful for peptide backbones include N-(2-aminoethyl)glycine monomers. Representative ligands include either the four main naturally occurring DNA bases (i.e., thymine, cytosine, adenine or guanine) or other naturally occurring nucleobases (e.g., inosine, uracil, 5-methylcytosine or thiouracil) or artificial bases (e.g., bromothymine, azaadenines or azaguanines, etc.) attached to a peptide backbone through a suitable linker. Alternatively, RNA bases can be substituted for the DNA bases. Suitable linkers include aza nitrogen atoms, such as those compositions and methods described in WO 92/20702, and additionally through amido and/or ureido tethers, such methylenecarbonyl linkers, or others as described in U.S. Pat. No. 5,539,082.

PNA molecules can be constructed with various peptide backbones, ligands, and linkers, and can be complexed with other molecules to enhance their binding affinity or resistance to nucleases. Many variations of PNA structure and methods of synthesis exist, and are well described in the art. All such PNA molecules and methods of synthesis are comprehended by the present invention, including those described in U.S. Pat. Nos. 5,539,082; 5,539,083; 5,641,625; 5,629,152; 5,700,922; 5,786,461; published PCT applications WO 92/20702, WO 92/20703, and WO 98/52595, and Egholn et al., “Peptide Nucleic Acids (PNA). Oligonucleotide Analogues with an Achiral Peptide Backbone,” J. Am. Chem. Soc., 114:1895-1897, 1992, all of which are fully incorporated by reference.

As compared to phosphorothioate oligodeoxynucleotides (PsODN), a well-studied DNA mimic, PNA has unique molecular characteristics that fortify its utility as a gene-intervening tool. These characteristics include a resistance to nucleases and proteases (Demidov et al., Biochem Pharmacol 48:1310-3, 1994), sequence specific hybridization to DNA or RNA target sequences via Watson-Crick base pair formation (Willey et al., J Virol 68:1029-39, 1994; and Freed et al., J Virol 68:5311-20, 1994), or Hoogsteen base pair formation, higher thermal stability of PNA/DNA (RNA) complex than corresponding DNA/DNA (RNA) complex, and a prompt destabilization of mismatched PNA/DNA (RNA) duplex (Egholm et al., Nature 365:566-8, 1993; Egholm et al., J. Am Chem Soc 114, 1895-7, 1992; and Igloi Proc Natl Acad Sci U S A 95:8562-7, 1998).

Another form of nuclease-resistant oligonucleotide analog, morpholino phosphorodiamidate oligomers, has been shown to effectively block translation of certain mRNA in cultured cells via RNase H-independent mechanism (Partridge et al., Antisense Nucleic Acid Drug Dev 6:169-75, 1996; Summerton and Weller, Antisense Nucleic Acid Drug Dev 7:187-95, 1997; Stein et al., Antisense Nucleic Acid Drug Dev 7:151-7, 1997; and Summerton et al., Antisense Nucleic Acid Drug Dev 7:63-70, 1997). The oligonucleotide analogs of the present invention can include morpholino phosphorodiamidate oligomers.

### Example 5

### Production of Protein Specific Binding Agents

Monoclonal or polyclonal antibodies may be produced to either a normal IGTP-family protein or mutant forms of such proteins. Optimally, antibodies raised against these proteins or peptides would specifically detect the protein or peptide with which the antibodies are generated. That is, an antibody generated to the IGTP protein, for instance, or a fragment thereof would recognize and bind the IGTP protein and would not substantially recognize or bind to other proteins found in human cells.

The determination that an antibody specifically detects an IGTP-family protein is made by any one of a number of standard immunoassay methods; for instance, the Western blotting technique (Sambrook et al., In Molecular Cloning: A Laboratory Manual, CSHL, New York, 1989). To determine that a given antibody preparation (such as one produced in a mouse) specifically detects the IGTP-family protein by Western blotting, total cellular protein is extracted from human cells (for example, lymphocytes) and electrophoresed on a sodium dodecyl sulfate-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase. Application of an alkaline phosphatase substrate 5-bromo-4-chloro-3-indolyl phosphate/nitro blue tetrazolium results in the production of a dense blue compound by immunolocalized alkaline phosphatase. Antibodies that specifically detect the IGTP-family protein will, by this technique, be shown to bind to the IGTP-family protein band (which will be localized at a given position on the gel determined by its molecular weight). Non-specific binding of the antibody to other proteins may occur and may be detectable as a weak signal on the Western blot The non-specific nature of this binding will be recognized by one skilled in the art by the weak signal obtained on the Western blot relative to the strong primary signal arising from the specific antibody-IGTP-family protein binding.

Substantially pure IGTP-family protein or protein fragment (peptide) suitable for use as an immunogen may be isolated from the transfected or transformed cells as described herein. Concentration of protein or peptide in the final preparation is adjusted, for example, by concentration on an Amicon filter device, to the level of a few micrograms per milliliter. Monoclonal or polyclonal antibody to the protein can then be prepared as follows:

A. Monoclonal Antibody Production by Hybridoma Fusion

Monoclonal antibody to epitopes of an IGTP-family protein can be prepared from murine hybridomas according to the classical method of Kohler and Milstein (Nature 256:495-497, 1975) or derivative methods thereof. Briefly, a mouse is repetitively inoculated with a few micrograms of the selected protein over a period of a few weeks. The mouse is then sacrificed, and the antibody-producing cells of the spleen isolated. The spleen cells are fused by means of polyethylene glycol with mouse myeloma cells, and the excess un-fused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Meth. Enzymol. 70:419-439, 1980), and derivative methods thereof. Selected positive clones can be expanded and their monoclonal antibody product harvested for use. Detailed procedures for monoclonal antibody production are described in Harlow and Lane (Antibodies, A Laboratory Manual, CSHL, New York, 1988).

B. Polyclonal Antibody Production by Immunization

Polyclonal antiserum containing antibodies to heterogenous epitopes of a single protein can be prepared by immunizing suitable animals with the expressed protein (Example 3), which can be unmodified or modified to enhance immunogenicity. Effective polyclonal antibody production is affected by many factors related both to the antigen and the host species. For example, small molecules tend to be less immunogenic than others and may require the use of carriers and adjuvant. Also, host animals vary in response to site of inoculations and dose, with either inadequate or excessive doses of antigen resulting in low titer antisera. Small doses (ng level) of antigen administered at multiple intradermal sites appear to be most reliable. An effective immunization protocol for rabbits can be found in Vaitukaitis et al. (J. Clin. Endocrinol. Metab. 33:988-991, 1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony et al. (In Handbook of Experimental Immunology, Wier, D. (ed.) chapter 19, published by Blackwell, 1973). Plateau concentration of antibody is usually in the range of about 0.1 to 0.2 mg/ml of serum (about 12 μM). Affinity of the antisera for the antigen is determined by preparing competitive binding curves, as described, for example, by Fisher (Manual of Clinical Immunology, Ch. 42, 1980).

C. Antibodies Raised against Synthetic Peptides

A third approach to raising antibodies against the subject IGTP locus encoded proteins or peptides is to use one or more synthetic peptides synthesized on a commercially available peptide synthesizer based upon the predicted amino acid sequence of the IGTP locus encoded protein or peptide.

By way of example only, a representative polyclonal antibodies to specific an immunogenic peptides within IGTP has been generated and is described in Taylor et al. (J. Biol. Clem., 271:20399-20405, 1996).

Antibody preparations prepared according to these protocols are useful in quantitative immunoassays which determine concentrations of antigen-bearing substances in biological samples; they are also used semi-quantitatively or qualitatively to identify the presence of antigen in a biological sample; or for immunolocalization of the IGTP protein.

For administration to human patients, antibodies, e.g., IGTP specific monoclonal antibodies, can be humanized by methods known in the art. Antibodies with a desired binding specificity can be commercially humanized (Scotgene, Scotland, UK; Oxford Molecular, Palo Alto, Calif.).

### Example 6

### Nucleic Acid-Based Diagnosis

One application of the herein-disclosed assignment of function to IGTP-family proteins is in the area of genetic testing for predisposition to infection, in particular parasitic and/or bacterial infections that are linked to defective (abnormal) IGTP-family proteins. Individuals carrying a mutation in an IGTP-family gene, or having amplifications or heterozygous or homozygous deletions of one or members of the IGTP-family, may be detected at the nucleic acid level with the use of a variety of techniques. For such a diagnostic procedure, a biological sample of the subject, which biological sample contains either DNA or RNA derived from the subject, is assayed for a mutated, amplified or deleted IGTP-family protein encoding sequence(s). Suitable biological samples include samples containing genomic DNA or RNA obtained from subject body cells, such as those present in peripheral blood, urine, saliva, tissue biopsy, surgical specimen, amniocentesis samples and autopsy material. The detection in the biological sample of a mutant IGTP-family gene, a mutant IGTP-family RNA, or an amplified or homozygously or heterozygously deleted IGTP-family gene, may be performed by a number of methodologies. Homology between IGTP-family genes in different species is advantageous because it allows nucleic acid probes having a sequence of one species to be used as a probe for the homologous gene in another species.

A. Detection of Unknown Mutations:

Unknown mutations can be identified through polymerase chain reaction amplification of reverse transcribed RNA (RT-PCR) or DNA isolated from cells of the subject, followed by direct DNA sequence determination of the products; single-strand conformational polymorphism analysis (SSCP) (for instance, see Hongyo et al., Nucleic Acids Res. 21:3637-3642, 1993); chemical cleavage (including HOT cleavage) (Bateman et al., Am. J. Med. Genet. 45:233-240, 1993; reviewed in Ellis et al., Hum. Mutat. 11:345-353, 1998); denaturing gradient gel electrophoresis (DGGE), ligation amplification mismatch protection (LAMP); or enzymatic mutation scanning (Taylor and Deeble, Genet. Anal. 14:181-186, 1999), followed by direct sequencing of amplicons with putative sequence variations.

B. Detection of Known Mutations:

Once one or more mutations within the IGTP gene are identified, the detection of specific known DNA mutations may be achieved by methods such as hybridization using allele specific oligonucleotides (ASOs) (Wallace et al., CSHL Symp. Quant. Biol. 51:257-261, 1986), direct DNA sequencing (Church and Gilbert, Proc. Natl. Acad. Sci. USA 81:1991-1995, 1988), the use of restriction enzymes (Flavell et al., Cell 15:25, 1978; Geever et al., 1981), discrimination on the basis of electrophoretic mobility in gels with denaturing reagent (Myers and Maniatis, Cold Spring Harbor Symp. Quant. Biol. 51:275-284, 1986), RNase protection (Myers et al., Science 230:1242, 1985), chemical cleavage (Cotton et al., Proc. Natl. Acad. Sci. USA 85:4397-4401, 1985), and the ligase-mediated detection procedure (Landegren et al., Science 241:1077, 1988). Oligonucleotides specific to normal or mutant IGTP sequences can be chemically synthesized using commercially available machines. These oligonucleotides can then be labeled radioactively with isotopes (such as ³²P) or non-radioactively, with tags such as biotin (Ward and Langer et al., Proc. Natl. Acad. Sci. USA 78:6633-6657, 1981), and hybridized to individual DNA samples immobilized on membranes or other solid supports by dot-blot or transfer from gels after electrophoresis. These specific sequences are visualized by methods such as autoradiography or fluorometric (Landegren et al., Science 242:229-237, 1989) or colorimetric reactions (Gebeyehu et al., Nucleic Acids Res. 15:4513-4534, 1987). Using an ASO specific for a normal allele, the absence of hybridization would indicate a mutation in the particular region of the gene, or deleted IGTP gene. In contrast, if an ASO specific for a mutant allele hybridizes to a clinical sample then that would indicate the presence of a mutation in the region defined by the ASO.

C. Detection of Genomic Amplification or Deletion:

Gene dosage (copy number) can have a strong influence on protein expression level; it is therefore advantageous to determine the number of copies of IGTP-family member-encoding nucleic acids in samples of subject cells or tissue. Probes generated from the IGTP-family member encoding sequence (GenBank Accession Number U53219, IGTP probes or primers; Accession Number M63630, IRG-47 probes or primers; Accession Number U19119, LRG-47 probes or primers; and so forth), or the reverse complement of the IGTP-family member encoding sequence, can be used to investigate and measure genomic dosage of the corresponding IGTP-family gene. Appropriate techniques for measuring gene dosage are known in the art; see for instance, U.S. Pat. No. 5,569,753 (“Cancer Detection Probes”) and Pinkel et al. (Nat. Genet. 20:207-211, 1998) (“High Resolution Analysis of DNA Copy Number Variation using Comparative Genomic Hybridization to Microarrays”).

Overexpression of an IGTP-family gene can also be detected by measuring the cellular level of the corresponding IGTP-family member-specific mRNA. mRNA can be measured using techniques well known in the art, including for instance Northern analysis, RT-PCR and mRNA in situ hybridization.

### Example 7

### Protein-Based Diagnosis

An alternative method of diagnosing abnormal an IGTP-family member is to quantitate the level of IGTP-family protein in the cells of an individual. This diagnostic tool would be useful for detecting reduced levels of the IGTP-family protein that result from, for example, mutations in the promoter regions of the corresponding IGTP-family gene or mutations within the coding region of the gene that produced truncated, non-functional or unstable polypeptides, as well as from deletions of a portion of or the entire IGTP-family gene.

Alternatively, genomic duplications of an IGTP-family locus may be detected as an increase in the expression level of an IGTP-family protein. Such an increase in protein expression may also be a result of an up-regulating mutation-in the promoter region or other regulatory or coding sequence within the corresponding IGTP-family gene. Localization and/or coordinated IGTP expression (temporally or spatially) can also be examined using well known techniques. The determination of reduced or increased IGTP-family member expression, in comparison to such expression in a normal cell, would be an alternative or supplemental approach to the direct determination of an IGTP-family member encoding sequence deletion, amplification or mutation status by the methods outlined above and equivalents.

The availability of antibodies specific to IGTP-family proteins will facilitate the detection and quantitation of cellular IGTP-family proteins by one of a number of immunoassay methods which are well known in the art and are presented in Harlow and Lane (Antibodies, A Laboratory Manual, CSHL, New York, 1988). Methods of constructing such antibodies are discussed above, in Example 5.

Any standard immunoassay format (e.g., ELISA, Western blot, or RIA assay) can be used to measure IGTP-family protein levels; comparison is usually to wild-type (normal) levels of the corresponding IGTP-family member. A decrease or substantial increase in IGTP-family polypeptide is indicative of an abnormal biological condition such as increased susceptibility to infection (e.g., bacterial or parasitic infection, such as by L. monocytogenes or T. gondii, respectively).

For the purposes of quantitating an IGTP protein, a biological sample of the subject, which sample includes cellular proteins, is required. Such a biological sample may be obtained from body cells, such as those present in peripheral blood, urine, saliva, tissue biopsy, amniocentesis samples, surgical specimens and autopsy material. Cells of the immune system may be of particular interest. Quantitation of IGTP protein may be achieved by iumunoassay and compared to levels of the protein found in healthy cells. A significant (e.g., 30% or greater) reduction in the amount of IGTP. protein in the cells of a subject compared to the amount of IGTP protein found in normal human cells could be assigned as an indication that the subject may have deletions or down-regulating mutations in the IGTP gene, whereas a significant (e.g., 30% or greater) increase would indicate that a duplication (amplification) or up-regulation may have occurred. Substantial under- or over-expression of IGTP protein may be indicative of an increased susceptibility of the subject to infection, more particularly to parasite (e.g., protozoan or helminth) infection.

### Example 8

### IGTP Knockout and Overexpression Transgenic Animals

Mutant organisms that under-express or over-express one or more IGTP-family proteins are useful for research, and to screen candidate compounds for anti-infectious agent (e.g., anti-protozoan and/or anti-bacterial) activity. Such mutants allow insight into the physiological and/or pathological role of IGTP-family members in a healthy and/or pathological organism. These mutants are “genetically engineered,” meaning that information in the form of nucleotides has been transferred into the mutant's genome at a location, or in a combination, in which it would not normally exist Nucleotides transferred in this way are said to be “non-native.” For example, a non-IGTP promoter inserted upstream of a native IGTP gene would be non-native. An extra copy of an IGTP-family gene on a plasmid, transformed into a cell, also would be non-native.

Mutants may be, for example, produced from mammals, such as mice, that either over-express or under-express an IGTP-family protein, or that do not express a particular IGTP-family protein at all. Over-expression mutants may be made by increasing the number of a specific gene (e.g., IGTP, LRG-47, IRG-47, and so forth) in the organism, or by introducing an IGTP-family gene into the organism under the control of a constitutive or inducible or promoter (e.g., a viral promoter) such as the mouse mammary tumor virus (MMTV) promoter or the whey acidic protein (WAP) promoter or the metallothionein promoter. Mutants that under-express an IGTP-family protein may be made by using an inducible or repressible promoter, or by deleting the corresponding IGTP-family gene, or by destroying or limiting the function of the IGTP-family gene, for instance by disrupting the gene by insertional mutagenesis.

Antisense genes may be engineered into the organism, under a constitutive or inducible promoter, to decrease or prevent IGTP-family protein expression.

A gene is “functionally deleted” when genetic engineering has been used to negate or reduce gene expression to negligible levels. When a mutant is referred to in this application as having an IGTP-family gene altered or functionally deleted, this refers to the IGTP-family gene and to any ortholog of this gene. When a mutant is referred to as having “more than the normal copy number” of a gene, this means that it has more than the usual number of genes found in the wild-type organism, e.g., in the diploid mouse or human

A mutant mouse over-expressing an IGTP-family protein may be made by constructing a plasmid having the IGTP-family gene driven by a promoter, such as the mouse mammary tumor virus (MMTV) promoter or the whey acidic protein (WAP) promoter. This plasmid may be introduced into mouse oocytes by microinjection. The oocytes are implanted into pseudopregnant females, and the litters are assayed for insertion of the transgene. Multiple strains containing the transgene are then available for study.

WAP is quite specific for mammary gland expression during lactation, and MMTV is expressed in a variety of tissues including mammary gland, salivary gland and lymphoid tissues. Many other promoters might be used to achieve various patterns of expression, e.g., the metallothionein promoter.

An inducible system may be created in which the subject expression construct is driven by a promoter regulated by an agent that can be fed to the mouse, such as tetracycline. Such techniques are well known in the art.

It will be realized that transgenic animals can be produced that have more than one changed IGTP-family protein, for instance in which two or more IGTP-family proteins are Imocked out, or over-expressed. Alternatively, in the same transgenic animal, one (or more) IGTP-family protein can be knocked out while another is over-expressed.

A mutant transgenic knockout animal (e.g., mouse) from which an IGTP-family gene is deleted can be made by removing coding regions of the specific IGTP-family gene (e.g., IGTP, LRG-47, IRG-47, and so forth) from embryonic stem cells. The methods of creating deletion mutations by using a targeting vector have been described (Porter and Sande, New Engl. J. Med., 327:1643-1648, 1992; U.S. Pat. Nos. 5,616,491, 5,981,830, 5,814,318, and 5,955,644, herein incorporated by reference).

By way of example only, and to provide experimental detail, knockout mice that are deficient in IGTP, LRG-47, or IRG-47 protein production can be and have been generated as described above (Section II).

### Example 9

### Kits

Kits are provided that contain the necessary reagents for detecting changes in the nucleic acid(s) that encode an IGTP-family protein, such as probes or primers specific for the IGTP gene or specific for identified mutations within this gene. Kits are also provided to determine altered expression (e.g., under- or overexpression) of an IGTP-family protein (i.e., containing antibodies or other IGTP-family-protein specific binding agents). Such kits also may include written instructions. The instructions can provide calibration curves or charts to compare with the determined (e.g., experimentally measured) values. The kits can also include control reagents, such as probes or primers for nucleotide sequence that would not be expected to be affected by alteration of the IGTP-family member. Kits for use in treatment and/or prevention of microbial diseases are also provided.

A. Kits For Detection of Changes in IGTP-family Member-Encoding Nucleic Acids

With the provision herein of the function of IGTP-family proteins, such as IGTP, IRG-47, and LRG-47, this information can now be used to generate kits for use in detection of abnormal IGTP-family encoding nucleic acids, and for detecting the susceptibility of a subject to infection(s) (e.g., bacterial and/or parasitic infection) mediated by an abnormal IGTP-family member. In such a kit, an amount of one or more oligonucleotide probes and/or primers, specific for an IGTP-family member, is provided in one or more containers. The oligonucleotide(s) may be provided suspended in an aqueous solution or as a freeze-dried or lyophilized powder, for instance. The container(s) in which the oligonucleotide(s) are supplied can be any conventional container that is capable of holding the supplied form, for instance, microfuge tubes, ampoules, or bottles. In some applications, pairs of primers may be provided in pre-measured single use amounts in individual, typically disposable, tubes or equivalent containers, in particular for use in amplification (e.g., PCR) reactions. With such an arrangement, the sample to be tested for the presence of an abnormal IGTP-family member nucleic acid can be added to the individual tubes and in vitro amplification (e.g., PCR) carried out directly.

For instance, Northern blot analysis to measure the amount of an IGTP-family mRNA, by way of example murine IGTP, could be carried out using a probe constructed from the 0.28-kb EcoRI fragment of the IGTP cDNA (residues 1645-1927 of the nucleotide sequence of GenBank Accession Number U53219) (Taylor et al., J. Biol. Chem., 271:20399-20405, 1996). For kits that employ PCR or other nucleic acid amplification techniques, such as one used to detect IGTP encoding sequences, such sequences may be amplified theoretically using the following combination of primers:

primer 1: residues 348-365 of the nucleotide sequence of GenBank Accession Number U53219; and

primer 2: the reverse complement of residues 1619-1602 of the nucleotide sequence of GenBank Accession Number U53219.

These primers are illustrative only; one skilled in the art will appreciate that many different primers may be derived from the provided cDNA sequence in order to amplify particular regions of these cDNAs.

The amount of each oligonucleotide probe or primer supplied in the kit can be any appropriate amount, depending for instance on the market to which the product is directed. For instance, if the kit is adapted for research or clinical use, the amount of each oligonucleotide primer provided would likely be an amount sufficient to prime several in vitro amplification reactions, or the amount of a probe provided would likely be sufficient to hybridize in several reactions. Those of ordinary skill in the art know the amount of oligonucleotide primer or probe that is appropriate for use in a single amplification or hybridization reaction. General guidelines may for instance be found in Innis et al. (PCR Protocols, A Guide to Methods and Applications, Academic Press, Inc., San Diego, Calif., 1990), Sambrook et al. (In Molecular Cloning: A Laboratory Manual, Cold Spring Harbor, N.Y., 1989), and Ausubel et al. (In Current Protocols in Molecular Biology, John Wiley & Sons, New York, 1998).

A kit may include more than two primers, in order to facilitate the PCR in vitro amplification of IGTP-family member encoding sequences.

In some embodiments of the current invention, kits may also include the reagents necessary to carry out hybridization reaction or in vitro amplification reactions, including, for instance, DNA sample preparation reagents, appropriate buffers (e.g., wash or polymerase buffer), salts (e.g., magnesium chloride), and deoxyribonucleotides (dNTPs). Written instructions may also be included.

Kits may in addition include either labeled or unlabeled oligonucleotide probes for use in detection of the in vitro amplified encoding sequences. The appropriate sequences for such a probe will be any sequence that falls between the annealing sites of the two provided oligonucleotide primers, such that the sequence the probe is complementary to is amplified during the in vitro amplification reaction.

It may also be advantageous to provided in the kit one or more control sequences for use in the PCR or hybridization reactions. The design of appropriate positive control sequences is well known to one of ordinary skill in the appropriate art. An example of such a control sequence would be a portion of glyceraldehyde-3-phosphate dehydrogenase (GADPH) or β-actin for example, as shown in Taylor et al. (J. Biol. Chem., 271:20399-20405, 1996).

B. Kits For Detection of Changes in Protein Expression

Kits for the detection of changes in protein expression (e.g., overexpression) of one or more IGTP-family proteins are also encompassed in the current invention. Such kits will include at least one IGTP-family protein specific binding agent (e.g., a polyclonal or monoclonal antibody or antibody fragment that retains specific binding ability) and may include at least one control. The IGTP-family protein specific binding agent and control may be provided in separate containers. The kits may also include a means for detecting target protein:agent complexes, for instance the agent may be detectably labeled. If the detectable agent is not labeled, it may be detected by second antibodies or protein A for example which may also be provided in some kits in one or more separate containers. Such techniques are well known.

Additional components in some kits include instructions for carrying out the assay. Instructions will allow the tester to determine whether IGTP-family protein expression levels are altered, e.g., elevated. Reaction vessels and auxiliary reagents such as chromogens, buffers, enzymes, etc. may also be included in the kits.

C. Kits for Treatment/Prevention of Microbial Infection

Kits for the treatment and/or prevention of one or more microbial infections are also provided herein. Such kits will include at least one IGTP-family protein or protein encoding-sequence. In such a kit, a clinically effective amount of one or more of such proteins or encoding sequences, or a fragment, derivative, analog, or mimetic thereof, is provided in one or more containers. The therapeutic compound may be provided suspended in an aqueous or other solution or as a freeze-dried or lyophilized powder, for instance. In certain embodiments, the compounds or peptides will be provided in the form of a pharmaceutical composition.

Kits according to this invention can also include instructions, usually written instructions, to assist the user in treating a disorder, condition or disease (e.g., a disease mediated by a microbial organisms, such as a bacterium, virus, or parasite such as a protozoan) with an IGTP-family protein or protein encoding sequence. Such instructions can optionally be provided on a computer readable medium.

The container(s) in which the protein(s) and/or encoding nucleic acid(s) are supplied can be any conventional container that is capable of holding the supplied form, for instance, microfuge tubes, ampoules, or bottles. In some applications, the therapeutic compound or peptide may be provided in pre-measured single use amounts in individual, typically disposable, tubes or equivalent containers.

The amount of a protein or encoding nucleic acid supplied in the kit can be any appropriate amount, depending for instance on the market to which the product is directed. For instance, if the kit is adapted for research or clinical use, the amount of each IGTP-family protein or protein encoding nucleic acid provided would likely be an amount sufficient for several treatments.

Certain kits according to this invention will also include one or more other agents useful in inhibiting microbial infection, e.g., an anti-viral, anti-bacterial, or anti-parasitic compound.

### Example 10

### Incorporation of IGTP-family Protein(s) or Encoding Nucleic Acid Molecules into Pharmaceutical Compositions

Pharmaceutical compositions that comprise at least one IGTP-family protein or encoding nucleic acid molecule, for instance murine or human IGTP protein or cDNA, as an active ingredient will normally be formulated with an appropriate solid or liquid carrier, depending upon the particular mode of administration chosen. The pharmaceutically acceptable carriers and excipients useful in this invention are conventional. For instance, parenteral formulations usually comprise injectable fluids that are pharmaceutically and physiologically acceptable fluid vehicles such as water, physiological saline, other balanced salt solutions, aqueous dextrose, glycerol or the like. Excipients that can be included are, for instance, other proteins, such as human serum albumin or plasma preparations. If desired, the pharmaceutical composition to be administered may also contain minor amounts of non-toxic auxiliary substances, such as wetting or emulsifying agents, preservatives, and pH buffering agents and the like, for example sodium acetate or sorbitan monolaurate.

Other medicinal and pharmaceutical agents, for instance anti-infectious agents such as anti-bacterial, anti-viral, or anti-parasitic agents, also may be included. In particular embodiments of the invention, it will be advantageous to include one or more conventional (e.g., chemical) anti-protozoan agents in an IGTP, LRG-47, or IRG-47-containing pharmaceutical composition. Likewise, in particular embodiments of the invention, it will be advantageous to include one or more conventional anti-bacterial agents in an LRT-47-containing pharmaceutical composition. Conventional anti-infectious agents are well known to those of ordinary skill in the art, and include for instance pyrimethamine and sulfonamide compounds. (See, e.g., Part XXII, Cecil Textbook of Medicine, Wyngaarden et al. (ed.), W. B. Saunders Co., Philadelphia, Pa., 1992, for information on conventional dosages and other information about such agents.)

IGTP, LRG-47, and IRG-47 are intracellular proteins, localizing predominantly to the ER. This localization indicates that it would be beneficial to apply protein-based pharmaceutical compounds of this invention to subjects in a manner specifically tailored to the delivery of proteins to the inside of cells. For example, a nucleic acid encoding the IGTP, or related protein, or another IGTP-family protein, is applied directly to the cells of a subject, such that the pharmaceutical protein is expressed within the target cell. Examples of such techniques direct application of naked DNA, or a “DNA vaccine” (see for instance Gregoriadis, Pharm. Res. 15, 616-670, 1998 for a review of such techniques, or Asakura et al., Clin. Exp. Immunol. 119:130-139, 2000; Yang et al., Int. J. Cancer, 83:532-540, 1999; McCluskie et al., Antisense Nucleic Acid Drug Dev., 8:401414, 1998, for recent developments in this technology). Alternatively, IGTP-family member encoding sequences can be inserted into cells and the cells used to treat the subject, a technique known to those of ordinary skill in the art.

Where it is advantageous to apply an IGTP-family protein rather than an encoding sequence, liposome technology can be employed. For a review of this technology, see Alving (Ann. NY Acad. Sci., 754:143-152, 1995); recent developments can be found in Hayashi et al. (Biochem. Biophys. Res. Commun., 261:824-828, 1999), Baca-Estrada et al. (J. IFN Cytokine Res., 19:455-462, 1999), and Babai et al. (Vaccine, 17:1239-1210, 1999). In addition, since many IGTP-family proteins are lipid-binding protein, it may be advantageous to supply the protein (or fragments or variants thereof) to a subject accompanied by one or more lipid carriers.

The dosage form of the pharmaceutical composition will be determined by the mode of administration chosen. For instance, in addition to injectable fluids, topical and oral formulations can be employed. Topical preparations can include eye drops, ointments, sprays and the like. Oral formulations may be liquid (e.g., syrups, solutions or suspensions), or solid (e.g., powders, pills, tablets, or capsules). For solid compositions, conventional non-toxic solid carriers can include pharmaceutical grades of mannitol, lactose, starch, or magnesium stearate. Actual methods of preparing such dosage forms are known, or will be apparent, to those skilled in the art.

The pharmaceutical compositions that comprise IGTP-family protein or encoding nucleic acid molecule maybe formulated in unit dosage form, suitable for individual administration of precise dosages. One possible unit dosage contains approximately 100 μg of protein, or about 50 μg of encoding nucleic acid molecule. The amount of active compound administered will be dependent on the subject being treated, the severity of the affliction, and the manner of administration, and is best left to the judgment of the prescribing clinician. Within these bounds, the formulation to be administered will contain a quantity of the active component(s) in an amount effective to achieve the desired effect in the subject being treated.

### Example 11

### Clinical Use of IGTP-Family Proteins and Encoding Nucleic Acids

The assignment herein of specific anti-infectious activities, more particularly an anti-protozoan and/or anti-bacterial activity, to specific IGTP-family proteins makes these proteins, and fragments, variants, analogs, derivatives or mimetic thereof that maintain immune response modifying activity, useful for treating infections in human and other animal subjects. Possibly susceptible infectious agents include parasites, for instance parasites (including protozoan and helminth parasites), bacteria, viruses, and fungi that infect various animals. Proteins and nucleic acids from IGTP will be particularly efficacious in reducing or preventing the infectivity of infectious agents that trigger a strong Th1 immune response in the infected animal.

In addition, use of an IGTP-family protein or encoding nucleic acids will also be beneficial in instances of auto-immune disease that are mediated by over- or under-active Th1 immune activity in a subject. In general, such auto-imnune disorders are those that involve an over-stimulation or mis-regulation of the Th1 immune response system, such as irritable bowel disease (IBD), rheumatoid arthritis, auto-immune diabetes mellitus, lupus erythematosis, sarcoidosis, multiple sclerosis, chronic delayed type hypersensitivity (DTH), and auto-immune encephalomyelitis. IGTP-family protein antagonists (such as monoclonal antibodies that bind to and inactivate IGTP) could, for example, be used to reduce an unwanted immune response.

The IGTP-family proteins of this invention may be administered to humans, or other animals on whose cells they are effective, in various manners such as topically, orally, intravenously, intramuscularly, intraperitoneally, intranasally, intradermally, intrathecally, and subcutaneously. The particular mode of administration and the dosage regimen will be selected by the attending clinician, taling into account the particulars of the case (e.g., the subject, the disease, and the disease state involved, and whether the treatment is prophylactic or post-infection). Treatment may involve daily or multi-daily doses of one or more IGTP-family protein(s) over a period of a few days to months, or evenyears.

IGTP, IRG-47, and LRG-47 are intracellular proteins, localizing predominantly to the ER. Liposome vaccine technology can be employed to deliver IGTP-family protein(s) to target cells, and more particularly to the interior compartment(s) of such target cells. See Alving (Ann. NY Acad. Sci., 754:143-152, 1995); Hayashi et al. (Biochem. Biophys. Res. Commun., 261:824-828, 1999), Baca-Estrada et al. (J. IFN Cytokine Res., 19:455-462, 1999), and Babai et al. (Vaccine, 17:1239-1210, 1999). In addition, since IGTP is a lipid-binding protein, it may be advantageous to supply the protein (or fragments or variants thereof) to a subject accompanied by one or more lipid carriers. If treatment is through the direct administration of cells expressing the IGTP protein to the subject,. such cells (e.g. transgenic pluripotent or hematopoietic stem cells or B cells) may be administered at a dose of between about 10⁶ and 10¹⁰ cells, on one or several occasions. The appropriate number of cells will depend on the patient, as well as the IGTP-family protein, or fragment or variant thereof, and cells chosen to express it.

Strategies for transferring genes into donor cells are well known, and for instance one such method is disclosed in U.S. Pat. No. 5,529,774. Generally, a gene encoding a protein (or fragment or variant thereof) having therapeutically desired effects is cloned into a viral expression vector, and that vector is then introduced into the target organism. The virus infects the cells, and produces the protein sequence in vivo, where it has its desired therapeutic effect. See also, Zabner et al., Cell 75:207-216, 1993.

As an alternative to adding the sequences encoding the IGTP-family protein or a homologous protein to the DNA of a virus, it is also possible to introduce such a gene into the somatic DNA of infected or uninfected cells, by methods that are well known in the art (Sambrook et al., Molecular Cloning: A Laboratory Manual, CSHL, New York, 1989). These methods can be used to introduce the functional IGTP molecules to human cells to provide long-term infection, and particularly Th1-response triggering infections (such as protozoan infection). Provision of the IGTP-family molecules such that they are expressed, as is the native sequence, upon exposure to IFNγ, will provide appropriate Th1-linked expression.

IGTP-family proteins, or pharmaceutically active fragments or variants thereof, are particularly useful in the prevention of infection during or immediately after exposure to bacteria or parasites (e.g., exposure to T. gondii or other infectious agents). In such instances, one or more doses of the pharmaceutical protein are administered immediately before or soon after exposure to the infectious agent. To prevent or ameliorate mother/infant transmission of parasite infection, for instance, it may be appropriate to administer the IGTP-family protein to the mother intermittently throughout pregnancy, and/or immediately before or following delivery, and/or directly to the newborn immediately after birth.

In addition to their individual use, an IGTP-family molecule as disclosed in the current invention may be combined with or used in association with other anti-microbial or anti-infectious pharmaceutical compounds, for instance anti-microbial (e.g., anti-bacterial and/or anti-protozoan) drugs, for providing therapy against infections or auto-immune conditions against which the IGTP-family molecule is effective. Such compounds can include, for instance, pyrimethamine sulfonamide (e.g., sulfamerazine, sulfadiazine, suflasoxazole, sulfamethazine, or sulfadiazine) or mixtures thereof. Dosages and formulations of such compounds are well known (see, for instance, Cecil Textbook of Medicine, Wyngaarden et al. (ed.), W. B. Saunders Co., Philadelphia, Pa., 1992).

It may also be advantageous to include other immune stimulators, for instance those that stimulate a strong Th1 response (such as IFNγ), in such pharmaceutical compositions.

### Example 12

### Use of IGTP-family Proteins as Adjuvants

The infection-related immune response modulating activity exhibited by IGTP-family proteins makes these proteins, and fragments, variants, analogs, derivatives, and mimetics thereof, useful as adjuvants for the enhancement of infection-related immune response in a subject. IGTP-family member adjuvant activity is useful in enhancing the response of a subject to various antigens derived from infectious agents. By way of example, antigens derived from infectious agents that are useful for co-administration or sequential administration with the disclosed molecules include parasite-derived antigens such as those disclosed in the following patent documents: U.S. Pat. No. 5,686,080 (helminth antigens); U.S. Pat. No. 5,804,200 (nematode antigens); U.S. Pat. No. 5,935,583 (Giardia antigens); U.S. Pat. No. 5,578,453 (protozoan, particularly T. gondii, antigens); U.S. Pat. No. 5,859,196 (protozoan, particularly T. gondii, antigens); and published PCT applications WO 99/45957 (various infectious agents) and WO 98/06434 (T. gondii antigens). Likewise, antigens derived from bacterial infectious agents are known, and can be co- or sequentially adminsistered with the disclosed molecules (see, e.g., U.S. Pat. No. 5,961,985 (Enterobacteriaceae antigens), U.S. Pat. No. 6,127,151 (Bordetella antigens), and U.S. Pat. No. 5,770,208 (Staphyolococcal antigens) for examples).

The adjuvant IGTP-family molecules of the invention will be employed in conventional fashion, and using an amount equivalent to that used in conventional, immune-modulatory protein-based adjuvant methods. See U.S. Pat. No. 5,980,912 (chitosan as an adjuvant) and U.S. Pat. No. 5,985,264 (IL-12 as an adjuvant), and published PCT applications WO 97/42969 (interleukin and interferon as adjuvants) and WO 98/33517 (interferon as an adjuvant).

In view of the many possible embodiments to which the principles of the invention may be applied, it should be recognized by one of ordinary skill in the relevant art that the illustrated embodiments are examples of the invention, and should not be taken as a limitation on the scope of the invention. Rather, the scope of the invention is defined by the following claims. We therefore claim as our invention all that comes within the scope and spirit of these claims.

We claim:

1. A method of modifying an anti-microbial immune response in a subject, comprising modifying an IGIP-family protein activity in the subject to enhance or inhibit the immune response.

2. The method of claim 1, wherein modifying the IGTP-family protein activity comprises altering expression of at least one IGTP-family protein.

3. The method of claim 1, wherein modifying the IGTP-family protein activity comprises supplying the subject with exogenous IGTP-family protein, or a fragment, variant, analog, derivative or mimetic thereof that maintains immune response modifying activity, or with a nucleic acid that encodes the IGTP-family protein, fragment, variant, analog, derivative or mimetic thereof.

4. The method of claim 3, wherein the subject is an animal infected with a bacterial or parasitic disease, or at risk for infection with a bacterial or parasitic disease.

5. The method of claim 1, wherein modifying the IGTP-family protein activity comprises decreasing tissue concentration of an IGTP-family protein in the subject.

6. The method of claim 1, wherein the anti-microbial immune response is a Th1 immune response.

7. The method of claim 1, wherein the anti-microbial immune response is an immune response to a bacterial, protozoa or helminth infection.

8. The method of claim 7, wherein the protozoa comprises Toxoplasma gondii.

9. The method of claim 1, wherein modifying the immune response comprises enhancing the immune response.

10. The method of claim 1, wherein modifying the immune response comprises inhibiting the immune response.

11. The method of claim 1, wherein modifying the immune response is effective to treat or prevent a disease caused by a bacteria or a parasite.

12. The method of claim 1 wherein the animal is a human.

13. The method of claim 12, wherein the anti-microbial response is an anti-parasitic response.

14. The method of claim 15 wherein the animal is a human infected with an organism selected from the group consisting of Toxoplasma sp., Plasmodium sp., Tryapanosoma sp., Leishmania sp., Cryptosporidium sp., Giardia sp., Entamoeba sp., Trichomonas sp., Diphyllobothriun latum, Echinococcosis sp., Schistosoma sp., Wuchereria bancrofti, Brugia malayi and Oncliocerca volvulus.

15. The method of claim 12, wherein the anti-microbial response is an anti-bacterial response.

16. The method of claim 15 wherein the animal is a human infected with an organism selected from the group consisting of Streptococcus sp., Haemophilus influenzae, Klebsiella sp., Escherichia sp., Legionella sp., Mycoplasma sp., Pneumocystis carinii, Listeria, Corynebacterium sp., Staphylococcus sp., Serratia, Pseudomonas, Shigella, Vibrio, Hemophilus sp., Yersinia, Enterobacter, Mycobacteria, Chlamydiae, and rickettsiae organisms.

17. The method of claim 2, wherein IGTP-family protein expression is altered by expressing in the subject a recombinant genetic construct comprising a promoter operably linked to a nucleic acid molecule, wherein the nucleic acid molecule comprises at least 10 consecutive nucleotides of a nucleotide sequence that encodes the IGTP-family protein, and expression of the nucleic acid molecule changes expression of the IGTP-family protein.

18. The method of claim 17 wherein expressing the recombinant genetic construct increases the IGTP-family protein expression.

19. The method of claim 1, wherein the method comprises inhibiting replication or infectivity of an infectious agent in a subject, or treating or preventing infection of the subject by the infectious agent, comprising administering to the subject an amount of IGTP-family protein or encoding sequence, or a fragment, variant, analog or mimetic thereof, sufficient to inhibit infectious agent replication or infectivity.

20. The method of claim 19, wherein the infectious agent is a bacterium.

21. The method of claim 19, wherein the infectious agent is a parasite.

22. The method of claim 21, wherein the parasite is chosen from the group consisting of helminth organisms and protozoan organisms.

23. The method of claim 22, wherein the parasite is a protozoa.

24. The method of claim 23, wherein the parasite is T. gondii.

25. The method of claim 1, wherein the method comprises providing enhanced immunogenicity of an antigen which evokes an immune response, comprising administering to a subject the antigen and an IGTP-family protein, or a fragment, variant, analog or mimetic thereof.

26. The method of claim 25, wherein the antigen is a bacterial antigen.

27. The method of claim 25, wherein the antigen is a protozoan antigen.

28. The method of claim 25, wherein the antigen is a Toxoplasma antigen.

29. The method of claim 1, wherein the method comprises stimulating an immune response to an anti-microbial immunogen, comprising co-administering with the immunogen an adjuvant effective amount of an IGTP-family protein, or a fragment, variant, analog or mimetic thereof, which retains IGTP-family protein adjuvant activity.

30. The method of claim 29, comprising co-administering the IGTP-family protein variant with the immunogen.

31. The method of claim 29, wherein co-administering IGTP-family protein, or the fragment, variant, analog or mimetic thereof, enhances the immune response beyond that achieved by administration of the anti-microbial immunogen alone.

32. The method of claim 1, wherein the method comprises detecting susceptibility of a subject to microbial infection, comprising detecting abnormal IGTP-family protein, abnormal IGTP-family protein expression, or abnormal IGTP-family protein-encoding nucleic acid in the subject.

33. The method of claim 32, wherein detecting abnormal IGTP-family protein expression in a subject comprises detecting an abnormally low level of IGTP, LRG-47, and/or IRG-47 in the subject.

34. The method of claim 32, wherein detecting abnormal IGTP-family protein expression in a subject comprises binding an oligonucleotide to an IGTP-family protein encoding sequence from the subject.

35. The method of claim 34, wherein the oligonucleotide comprises at least 10 consecutive nucleotides from the nucleic acid sequence of GenBank Accession Number U53219, M63630, or U19119.

36. The method of claim 32, wherein detecting abnormal IGTP expression in a subject comprises detecting an IGTP-family protein from the subject with an IGTP-family protein-specific protein binding agent.

37. The method of claim 36, wherein the binding agent is an antibody.

38. A kit for use with the method of claims 32-37, comprising at least one IGTP-family protein specific protein binding agent.

39. The kit of claim 38, wherein the binding agent is an IGTP-specific antibody, and LRG-47-specific antibody, or an IRG-47-specific antibody.

40. A kit for use with the method of claim 32-37, comprising at least one IGTP-family protein encoding nucleic acid specific binding agent.

41. The kit of claim 40, wherein the agent comprises at least 10 nucleotides from the nucleic acid sequence of GenBank Accession Number U53219, M63630, or U19119.

42. The kit of claim 40, wherein the binding agent is capable of specifically binding to at least a portion of
(a) the nucleic acid sequence of GenBank Accession Number U53219, M63630, or U19119;
(b) nucleic acid sequences that differ from those specified in (a) by one or more conservative amino acid substitutions; or
(c) nucleic acid sequences having at least 80% sequence identity to the sequences specified in (a) or (b).

43. A transgenic non-human animal in which IGTP-family member gene expression has been altered.

44. The transgenic non-human animal according to claim 43, wherein the IGTP-family member is IGTP, LRG-47, or IRG-47.

45. The transgenic non-human animal according to claim 43, wherein IGTP-family member is over-expressed relative to expression prior to IGTP-family member gene expression alteration.

46. The transgenic non-human animal according to claim 43, wherein IGTP-family member is under-expressed relative to expression prior to IGTP-family member gene expression alteration.

47. A method of screening for an anti-microbial compound, comprising administering a candidate compound to the transgenic animal of claim 43.

48. The method of claim 47, wherein the anti-microbial compound is an anti-bacterial compound.

49. The method of claim 47, wherein the anti-microbial compound is an anti-parasitic compound.

50. The method of claim 47, wherein the candidate compound comprises an anti-microbial drug.

51. The method of claim 48, wherein the anti-microbial drug is an analog of a recognized anti-protozoan or anti-bacterial drug.

52. A pharmaceutical composition comprising:
a pharmaceutically acceptable vehicle or carrier
a therapeutically effective amount of at least one anti-microbial compound; and
a therapeutically effective amount of at least one IGTP-family protein, or a fragment, variant, analog or mimetic thereof, or a nucleic acid that encodes an IGTP-family protein, fragment, variant, analog, derivative mimetic thereof.

53. The pharmaceutical composition of claim 52, wherein the anti-microbial compound is an anti-bacterial compound.

54. The pharmaceutical composition of claim 53, wherein the anti-bacterial compound is a bacterial antigen that evokes an immunogenic response to the bacterium.

55. The pharmaceutical composition of claim 52, wherein the anti-microbial compound is an anti-protozoan compound.

56. The pharmaceutical composition of claim 55, wherein the anti-protozoan compound is a protozoan antigen that evokes an immunogenic response to the protozoan.

57. The pharmaceutical composition of claim 52, wherein the anti-protozoan compound is an anti-microbial pharmaceutical compound.

58. The pharmaceutical composition of claim 57, wherein the anti-microbial pharmaceutical compound is a pharmaceutical compound effective in treating or preventing a Toxoplasma infection.

59. The pharmaceutical composition of claim 57, wherein the pharmaceutical compound is selected from the group consisting of pyrimethamine and a sulfonamide, or mixtures thereof.

60. The pharmaceutical composition of claim 59, wherein the sulfonamide is selected from the group consisting of sulfamerazine, sulfadiazine, suflasoxazole, sulfamethazine, and sulfadiazine, or mixtures thereof.

61. A kit comprising the pharmaceutical composition of any one of claims 52-60.
